SEQUENCE LISTING (2 sequences)

SEQ ID NO: 1 - protein, 537 aa, Mus Musculus
MASTRAKPTLPLLLALVTVVIPGPGDAQVSIHPREAFLPQGGSVQVNCSSSCKEDLSLGLETQWLKDELESGPNWKLFELSEIGEDSSPLCFENCGTVQSSASATITVYSFPESVELRPLPAWQQVGKDLTLRCHVDGGAPRTQLSAVLLRGEEILSRQPVGGHPKDPKEITFTVLASRGDHGANFSCRTELDLRPQGLALFSNVSEARSLRTFDLPATIPKLDTPDLLEVGTQQKLFCSLEGLFPASEARIYLELGGQMPTQESTNSSDSVSATALVEVTEEFDRTLPLRCVLELADQILETQRTLTVYNFSAPVLTLSQLEVSEGSQVTVKCEAHSGSKVVLLSGVEPRPPTPQVQFTLNASSEDHKRSFFCSAALEVAGKFLFKNQTLELHVLYGPRLDETDCLGNWTWQEGSQQTLKCQAWGNPSPKMTCRRKADGALLPIGVVKSVKQEMNGTYVCHAFSSHGNVTRNVYLTVLYHSQNNWTIIILVPVLLVIVGLVMAASYVYNRQRKIRIYKLQKAQEEAIKLKGQAPPP

SEQ ID NO: 2 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

(19) United States
(12) Patent Application Publication
(10) Pub. No.: US 2022/0280653 A1
(43) Pub. Date: Sep. 8, 2022

(54) INTERCELLULAR ADHESION MOLECULE 1 (ICAM1) ANTIBODY DRUG CONJUGATE AND USES THEREOF

(71) Applicant: Children's Medical Center Corporation, Boston, MA (US)

(72) Inventors: Marsha A. Moses, Brookline, MA (US); Jing Huang, Natick, MA (US); Peng Guo, Boston, MA (US)

(73) Assignee: Children's Medical Center Corporation, Boston, MA (US)

(21) Appl. No.: 17/637,305

(22) PCT Filed: Aug. 21, 2020
(86) PCT No.: PCT/US2020/047300

§ 371 Date: Feb. 22, 2022

Related U.S. Application Data
(60) Provisional application No. 62/891,170, filed on Aug. 23, 2019.

(51) Int. Cl. A61K 47/68; A61P 35/00; C07K 16/28; A61K 9/00

(52) U.S. Cl. CPC A61K 47/6859; A61K 47/6811; A61K 47/6849; A61P 35/00; C07K 16/2821; A61K 9/0019

(57) ABSTRACT

The disclosure provides compositions comprising intercellular adhesion molecule 1 (ICAM1) antibody and methods for using the same for therapeutic applications, for example, treating pancreatic cancer and predicting drug response.

### RELATED APPLICATION

This Application claims the benefit under 35 U.S.C. § 119(e) of U.S. Provisional Application Ser. No. 62/891,170, entitled “INTERCELLULAR ADHESION MOLECULE 1 (ICAM1) ANTIBODY DRUG CONJUGATE AND USES THEREOF” filed on Aug. 23, 2019, the entire contents of which are incorporated herein by reference.

### BACKGROUND

Pancreatic cancer (PC) remains one of the most lethal diseases and accounts for 56,770 people death in the United States within 2019, representing 7% of all cancer mortality. The prognosis for PC patients is strikingly poor with a 5 yr survival less than 8% despite of the recent intensified studies of immunotherapy and nanomedicine therapy.

### SUMMARY

The present disclosure is based, at least in part, on the surprising finding that intercellular adhesion molecule 1 (ICAM1) can be targeted to improve pancreatic cancer treatment and stratify patient populations for precision medicine. The immunosuppressive microenvironment of pancreatic cancer tumors presents several challenges to effective treatment. For example, the tumor microenvironment of pancreatic cancer tumors is often characterized by desmoplastic stroma and poor vascularization, which create physical barriers that prevent T-cells or drugs from efficiently infiltrating the tumors. These limitations are addressed, at least in part, by the present disclosure.

Provided herein, in some aspects, antibody-drug conjugates (ADCs) that comprise an intercellular adhesion molecule 1 (ICAM1), which are useful for treatment of pancreatic cancer. As described below, use of the ADCs comprising an ICAM1 antibody allowed for preferential targeting of pancreatic cancer cells over non-cancerous cells, which can improve the therapeutic window of drugs and limit toxicity. Predicting therapeutic sensitivity among patient populations is also challenging given the high genetic heterogeneity of pancreatic cancer. Accordingly, further aspects of the present disclosure provide methods of identifying patient populations for treatment with an ICAM1 antibody or an ADC comprising an ICAM1 antibody in a subject with pancreatic cancer.

Aspects of the present disclosure provide methods of treating pancreatic cancer comprising administering to a subject in need thereof an effective amount of an antibody drug conjugate (ADC) comprising an intercellular adhesion molecule 1 (ICAM1) antibody conjugated to a drug.

In some embodiments, the drug is selected from the group consisting of: N2′-Deacetyl-N2′-(3-mercapto-l-oxopropyl)mertansine (DM1), monomethyl auristatin E (MMAE), monomethyl auristatin F (MMAF), and duocarmycin. In some embodiments, the drug is DM1.

In some embodiments, the ICAM1 antibody and the drug is conjugated via a linker.

In some embodiments, the linker is a cleavable linker. In some embodiments, the cleavable linker is selected from the group consisting of: N-succinimidyl 4-(2-pyridyldithio)pentanoate (SPP), N-succinimidyl 3-(2-pyridyldithio)butanoate (SPDB), Sulfo-SPDB, valine-citrulline (Val-cit), acetyl butyrate, and CL2A. In some embodiments, the cleavable linker is Val-cit.

In some embodiments, the linker is a non-cleavable linker. In some embodiments, the non-cleavable linker is a selected from the group consisting of N-succinimidyl 4-(Nmaleimidomethyl)cyclohexane-1-carboxylate (SMCC) and maleimidomethyl cyclohexane-1-carboxylate (MCC). In some embodiments, the non-cleavable linker is a N-succinimidyl 4-(Nmaleimidomethyl)cyclohexane-1-carboxylate (SMCC) linker.

In some embodiments, the ICAM1 antibody is selected from the group consisting of an IgG, an Ig monomer, a Fab fragment, a F(ab′)2 fragment, a Fd fragment, a scFv, a scAb, a dAb, a Fv, an affibody, a diabody, a single domain heavy chain antibody, and a single domain light chain antibody.

In some embodiments, the ICAM1 antibody is Enlimomab or HCD54.

In some embodiments, the ratio of the ICAM1 antibody and the drug in the ADC is 1:1 to 1:10. In some embodiments, the ratio of the ICAM1 antibody and the drug in the ADC is 1:4.

In some embodiments, the ADC is administered via injection. In some embodiments, the injection is intravenous injection or intratumoral injection.

Further aspects of the present disclosure provide methods of treating pancreatic cancer comprising administering to a subject in need thereof an effective amount of an antibody drug conjugate (ADC) comprising an intercellular adhesion molecule 1 (ICAM1) antibody conjugated to N2′-Deacetyl-N2′-(3-mercapto-1-oxopropyl)mertansine (DM1) via a N-succinimidyl 4-(Nmaleimidomethyl)cyclohexane-1-carboxylate (SMCC) linker.

Further aspects of the present disclosure provide methods of predicting the responsiveness of treatment with an ICAM1 antibody or an antibody drug conjugate (ADC) comprising an intercellular adhesion molecule 1 (ICAM1) antibody conjugated to a drug in a subject having pancreatic cancer comprising: (i) administering to the subject an effective amount of an ICAM1 antibody labeled with an imaging agent; and (ii) visualizing the tumor via imaging; (iii) determining the level of ICAM1 on the tumor, wherein a higher level of ICAM1 indicates that the subject is more responsive to treatment with the ICAM1 antibody or the ADC compared to a subject having a lower level of ICAM1 (e.g., identifying the subject as being more responsive to the treatment if the level of ICAM1 is higher, compared to a subject with a tumor having a lower level of ICAM1).

In some embodiments, the ICAM1 antibody in (i) is labeled with DTPA-Gd.

In some embodiments, the visualizing in (ii) is via magnetic resonance imaging (MRI).

In some embodiments, the method further comprises administering an effective amount of the ICAM1 antibody or the ADC to the subject predicted to be responsive to the treatment to treat the pancreatic cancer.

In some embodiments, the drug is selected from the group consisting of: N2′-Deacetyl-N2′-(3-mercapto-1-oxopropyl)mertansine (DM1), monomethyl auristatin E (MMAE), monomethyl auristatin F (MMAF), and duocarmycin. In some embodiments, the drug is DM1.

In some embodiments, the ICAM1 antibody and the drug is conjugated via a linker.

In some embodiments, the linker is a cleavable linker. In some embodiments, the cleavable linker is selected from the group consisting of: N-succinimidyl 4-(2-pyridyldithio)pentanoate (SPP), N-succinimidyl 3-(2-pyridyldithio)butanoate (SPDB), Sulfo-SPDB, valine-citrulline (Val-cit), acetyl butyrate, and CL2A. In some embodiments, the cleavable linker is Val-cit.

In some embodiments, the linker is a non-cleavable linker. In some embodiments, the non-cleavable linker is a selected from the group consisting of N-succinimidyl 4-(Nmaleimidomethyl)cyclohexane-1-carboxylate (SMCC) and maleimidomethyl cyclohexane-1-carboxylate (MCC). In some embodiments, the non-cleavable linker is a N-succinimidyl 4-(Nmaleimidomethyl)cyclohexane-1-carboxylate (SMCC) linker.

In some embodiments, the ICAM1 antibody is selected from the group consisting of an IgG, an Ig monomer, a Fab fragment, a F(ab′)2 fragment, a Fd fragment, a scFv, a scAb, a dAb, a Fv, an affibody, a diabody, a single domain heavy chain antibody, and a single domain light chain antibody.

In some embodiments, the ICAM1 antibody is Enlimomab or HCD54.

In some embodiments, the ratio of the ICAM1 antibody and the drug in the ADC is 1:1 to 1:10.

In some embodiments, the ratio of the ICAM1 antibody and the drug in the ADC is 1:4.

Further aspects of the present disclosure provide an antibody drug conjugate (ADC) comprising an intercellular adhesion molecule 1 (ICAM1) antibody conjugated to a drug.

In some embodiments, the drug is selected from the group consisting of: N2′-Deacetyl-N2′-(3-mercapto-1-oxopropyl)mertansine (DM1), monomethyl auristatin E (MMAE), monomethyl auristatin F (MMAF), and duocarmycin. In some embodiments, the drug is DM1.

In some embodiments, the ICAM1 antibody and the drug is conjugated via a linker.

In some embodiments, the linker is a cleavable linker. In some embodiments, the cleavable linker is selected from the group consisting of: N-succinimidyl 4-(2-pyridyldithio)pentanoate (SPP), N-succinimidyl 3-(2-pyridyldithio)butanoate (SPDB), Sulfo-SPDB, valine-citrulline (Val-cit), acetyl butyrate, and CL2A. In some embodiments, the cleavable linker is Val-cit.

In some embodiments, the linker is a non-cleavable linker. In some embodiments, the non-cleavable linker is a selected from the group consisting of N-succinimidyl 4-(Nmaleimidomethyl)cyclohexane-1-carboxylate (SMCC) and maleimidomethyl cyclohexane-1-carboxylate (MCC). In some embodiments, the non-cleavable linker is a N-succinimidyl 4-(Nmaleimidomethyl)cyclohexane-1-carboxylate (SMCC) linker.

In some embodiments, the ICAM1 antibody is selected from the group consisting of an IgG, an Ig monomer, a Fab fragment, a F(ab′)2 fragment, a Fd fragment, a scFv, a scAb, a dAb, a Fv, an affibody, a diabody, a single domain heavy chain antibody, and a single domain light chain antibody.

In some embodiments, the ICAM1 antibody is Enlimomab or HCD54.

In some embodiments, the ratio of the ICAM1 antibody and the drug in the ADC is 1:1 to 1:10. In some embodiments, the ratio of the ICAM1 antibody and the drug in the ADC is 1:4.

Further aspects of the present disclosure provide an antibody drug conjugate (ADC) comprising an intercellular adhesion molecule 1 (ICAM1) antibody conjugated to N2′-Deacetyl-N2′-(3-mercapto-1-oxopropyl)mertansine (DM1) via a N-succinimidyl 4-(Nmaleimidomethyl)cyclohexane-1-carboxylate (SMCC) linker.

### BRIEF DESCRIPTION OF THE DRAWINGS

The accompanying drawings are not intended to be drawn to scale. In the drawings, each identical or nearly identical component that is illustrated in various FIGS. is represented by a like numeral. For purposes of clarity, not every component may be labeled in every drawing.

In the drawings:

FIGS. 1A-1H show that ICAM1 is differentially overexpressed in human PC tissues and cells. FIG. 1A is a heatmap of membrane proteins expression in human PC cells, compared with pancreatic normal epithelial cells. FIG. 1B is a Venn diagram showing the overlaps between the selected target sets for PC cells. FIG. 1C shows the top 10 upregulated surface proteins in PC cells, and ICAM1 expression level in human PC cell lines. FIG. 1D contains images showing IF staining of ICAM1 in human PC and normal pancreatic epithelial cells. FIG. 1E contains representative images of IHC staining of ICAM1 in human PC tumor tissues with different stages and normal pancreas tissues. FIG. 1F shows a comparison of ICAM1 IHC staining score between PC and normal tissues. FIG. 1G shows pathological scores for tumor microarrays correlated with TNM stages. FIG. 1H shows a Kaplan-Meier analysis of overall survival of 84 PC patients according to different ICAM1 levels. *P<0.05, **P<0.01, ***P<0.001.

FIGS. 2A-2G show that the ICAM1 antibody selectively recognizes and targets PC tumor in vivo. FIG. 2A is a schematic diagram of the orthotopic PC model injected PANC-1-LUC at Day 0, receiving ICAM-AF or IgG-AF 28 days post tumor inoculation (n=6 per group), and performing fluorescence imaging at Day 29. FIG. 2B shows ex vivo fluorescence imaging of PC tumors with surrounding normal pancreas tissues. FIG. 2C shows corresponding quantification of fluorescence intensity in tumors. FIG. 2D contains representative imaging flow cytometry images showing the PC-specific internalization of ICAM1 Ab in PANC-1, BxPC-3 and HPNE cells. FIG. 2E shows signal intensity analysis for ICAM1 antibody-mediated cell internalization. FIG. 2F shows cell proliferation of PANC-1 and BxPC-3 with treatment of ICAM1 Ab or IgG. FIG. 2G illustrates cell motion trajectories showing the response of PANC-1 and BxPC-3 after 24 h treatment of ICAM1 Ab. *P<0.05, **P<0.01, ***P<0.001.

FIGS. 3A-3L show that ICAM1-DM1 selectively ablates PC cells in vitro and in vivo. FIG. 3A is a schematic illustration of ICAM1-DM1 ADC. FIG. 3B shows results of screening of cytotoxic payload with different linkers that conjugated to ICAM1 Ab. FIGS. 3C-3E show results of cell viability assays to measure the anti-tumor activity of ICAM1-DM1 to PANC-1 (FIG. 3C) and BxPC-3 (FIG. 3D), and normal HPNE (FIG. 3E), compared with IgG-DM1 and GEM. FIG. 3F is a schematic diagram of the orthotopic PC model injected LUC-PANC-1-GFP at Week 0, receiving ICAM1-DM1, IgG-DM, gemcitabine or PBS 2 weeks post tumor inoculation (n=5,6 per group). IVIS was performed every week. FIG. 3G shows ex vivo fluorescence imaging of GFP-expressing PC tumors. FIG. 3H shows corresponding tumor diameters. FIG. 3I shows the total flux of bioluminescence of the tumors in different treatment groups. FIG. 3J contains images showing treatment of ICAM1-DM1 ADC inhibits tumor cell proliferation. FIG. 3K shows corresponding quantification of Ki67+ cell percentage. FIG. 3L shows that treatment of ICAM1-DM1 ADC inhibits metastasis. *P<0.05, **P<0.01, ***P<0.001.

FIGS. 4A-4C show results of non-invasive MRI to assess ICAM1-expressing PC tumor in vivo. FIG. 4A is a schematic diagram of the orthotopic PC model injected PANC-1-LUC at Day 0, receiving ICAM-Gd or IgG-Gd 28 days post tumor inoculation (n=3 per group), and performing MRI at Day29. FIG. 4B shows representative in vivo T1- and T2-weighted MR images of mice bearing orthotopic PC tumors received ICAM1-Gd and IgG-Gd. Tumor was circled and magnified in the insets. FIG. 4C shows quantitative changes of MRI signal-to-noise ratio in PC tumors. Bar graphs are shown as mean±SD. *P<0.05, **P<0.01, ***P<0.001.

FIG. 5 shows representative H&E staining of mouse organs from different treatment groups. Scale bar, 100 μm. Micrometastasis sites are indicated by large asterisks. Escalated lymphocytes in spleen are indicated by small asterisks.

FIG. 6 shows ICAM1-DM1 treatment reduced PDAC metastasis.

FIG. 7 shows ICAM1-DM1 has no long term toxicity in mice received treatment.

### DETAILED DESCRIPTION OF CERTAIN EMBODIMENTS

To date, pancreatic cancer (PC) remains among the most lethal diseases that accounts for 56,770 people death in the United States within 2019, representing 7% of all cancer mortality. The prognosis for PC patients is strikingly poor with a 5yr survival less than 8% despite of the recent intensified studies of immunotherapy and nanomedicine therapy. These undesirable results are largely due to the immunosuppressive tumor microenvironment (TME) of PC tumors, which is characterized by desmoplastic stroma and poor vascularization. Such TME creates physical barriers that prevent T-cells or nanomedicines efficiently infiltrating tumors and directly interacting with PC cells, leading to unfavorable efficacies. It highlights a critical need to develop novel targeted therapeutics that can better infiltrated PC tumors while maintaining potent tumor-specific efficacy.

Antibody drug conjugates (ADCs) have been shown to be promising clinical efficacy against several types of cancers including aggressive solid tumors like breast cancer, which response poorly to T-cell immunotherapy. Though several PC-targeted ADCs have been developed utilizing conventional PC targets (e.g., EGFR, EpHA2, and Mesothelin), there still lack a systematic and quantitative comparison of established PC targets and other candidates at their cell surface protein levels.

Provided herein, in some aspects, is an unbiased and quantitative screening of cell surface proteins to discover more optimal PC immunotherapeutic targets and promote the development of PC-targeted ADCs. ICAM1 was identified as a potential PC immunotherapeutic target. The ICAM1 ADC described herein induced potent and durable PC tumor regression in vivo. The present disclosure also envisions the use of ICAM1 as an immunotherapy target for pancreatic cancer, including, without limitation, T cell-based immunotherapies such as CART, and checkpoint blockade.

Further, the present disclosure provides a non-invasive MRI approach to identify ICAM1-expressing tumors suitable for ICAM1-targeting immunotherapy Such patients, in some embodiments, are administered the ICAM1 ADC for the treatment of the PC.

Aspects of the present disclosure provide antibody-drug conjugates (ADCs) comprising intercellular adhesion molecule 1 (ICAM1) and a drug, methods of using the same in treatment of pancreatic cancer, and methods of predicting the responsiveness of treatment with an ICAM1 antibody or ICAM1 antibody-drug conjugate (ADC).

### Antibody-Drug Conjugates (ADCs)

### I. ICAM1 Antibodies

Antibody-drug conjugates (ADCs) are a class of immunotherapeutics that comprise an antibody conjugated to a drug. The ADCs of the present disclosure can target cells expressing ICAM1. ICAM1 is a cell surface glycoprotein that has been shown to bind integrins of type CD11a/CD18, or CD11b/CD18 and has been implicated in mediating cell-cell interactions and promoting leukocyte endothelial transmigration. ICAM1 is also referred to as ICAM-1, BB2, Cluster of Differentiation 54 (CD54), and P3.58.

Non-limiting examples of amino acid sequences encoding ICAM1 include UniProtKB Accession Nos. P13597 and P05362.

UniProtKB Accession No. P13597 encodes ICAM1 from Mus Musculus has the sequence of:

(SEQ ID NO: 1)
MASTRAKPTLPLLLALVTVVIPGPGDAQVSIHPREAFLPQGGSVQVNCS
SSCKEDLSLGLETQWLKDELESGPNWKLFELSEIGEDSSPLCFENCGTV
QSSASATITVYSFPESVELRPLPAWQQVGKDLTLRCHVDGGAPRTQLSA
VLLRGEEILSRQPVGGHPKDPKEITFTVLASRGDHGANFSCRTELDLRP
QGLALFSNVSEARSLRTFDLPATIPKLDTPDLLEVGTQQKLFCSLEGLF
PASEARIYLELGGQMPTQESTNSSDSVSATALVEVTEEFDRTLPLRCVL
ELADQILETQRTLTVYNFSAPVLTLSQLEVSEGSQVTVKCEAHSGSKVV
LLSGVEPRPPTPQVQFTLNASSEDHKRSFFCSAALEVAGKFLFKNQTLE
LHVLYGPRLDETDCLGNWTWQEGSQQTLKCQAWGNPSPKMTCRRKADGA
LLPIGVVKSVKQEMNGTYVCHAFSSHGNVTRNVYLTVLYHSQNNWTIII
LVPVLLVIVGLVMAASYVYNRQRKIRIYKLQKAQEEAIKLKGQAPPP.

UniProtKB Accession No. P05362 encodes ICAM1 from Homo Sapiens and has the sequence:

(SEQ ID NO: 2)
MAPSSPRPALPALLVLLGALFPGPGNAQTSVSPSKVILPRGGSVLVTCS
TSCDQPKLLGIETPLPKKELLLPGNNRKVYELSNVQEDSQPMCYSNCPD
GQSTAKTFLTVYWTPERVELAPLPSWQPVGKNLTLRCQVEGGAPRANLT
VVLLRGEKELKREPAVGEPAEVTTTVLVRRDHHGANFSCRTELDLRPQG
LELFENTSAPYQLQTFVLPATPPQLVSPRVLEVDTQGTVVCSLDGLFPV
SEAQVHLALGDQRLNPTVTYGNDSFSAKASVSVTAEDEGTQRLTCAVIL
GNQSQETLQTVTIYSFPAPNVILTKPEVSEGTEVTVKCEAHPRAKVTLN
GVPAQPLGPRAQLLLKATPEDNGRSFSCSATLEVAGQLIHKNQTRELRV
LYGPRLDERDCPGNWTWPENSQQTPMCQAWGNPLPELKCLKDGTFPLPI
GESVTVTRDLEGTYLCRARSTQGEVTRKVTVNVLSPRYEIVIITVVAAA
VIMGTAGLSTYLYNRQRKIKKYRLQQAQKGTPMKPNTQATPP.

In some embodiments, a ICAM1 protein comprises a sequence that is at least 50%, 55%, 60%, 65%, 70%, 75%, 80%, 85%, 90%, 91%, 92%, 93%, 94%, 95%, 96%, 97%, 98%, 99%, or is 100% identical to SEQ ID NO: 1 or to SEQ ID NO: 2. Additional ICAM1 proteins are well known and may be identified using publically available databases including, e.g., GenBank. An ICAM1 protein may be from any species, including Homo sapiens.

Antibodies of the present disclosure are capable of binding ICAM1. In some embodiments, the ICAM1 antibody is a monoclonal antibody. In some embodiments, the ICAM1 antibody is a polyclonal antibody. In some embodiments, the ICAM1 antibody is a murine antibody. In some embodiments, the ICAM1 antibody is a humanized antibody.

Non-limiting examples of ICAM1 antibodies include clone HCD54 (“HCD54,” commercially available at BioLegend, catalog # 322702), UV3, RR1.1, R6.5 (BIRR-1 or Enlimomab, commercially available at Thermo Fisher Scientific, catalog # BMS1011) and BI-505. R6.5 (Enlimomab) is a monoclonal murine antibody produced by ATCC HB-9580 hybridoma cells, e.g., as described in U.S. Pat. No. 5,324,510, which is herein incorporated by reference.

UV3 is a monoclonal antibody and has been shown to bind to ICAM-1 on myeloma cells. In some embodiments, the ICAM1 antibody is a F(ab)′2 fragment of UV3. See, e.g., Huang et al., Hybridoma. 1993 December; 12(6): 661-75; and Coleman et al., J Immunother. 2006 September-October; 29(5): 489-98, which is each herein incorporated by reference. RR1.1 is a monoclonal ICAM1 antibody. See, e.g., Rothlein and Springer, 1986 J. Exp. Med. 163, 1132-1149, which is herein incorporated by reference. HCD54 is a monoclonal ICAM1 antibody. BI-505 is a fully human ICAM1 monoclonal antibody. See, e.g., Hansson et al., Clin Cancer Res. 2015 Jun. 15; 21(12): 2730-6, which is herein incorporated by reference.

The term “bind” refers to the association of two entities (e.g., two proteins). Two entities (e.g., two proteins) are considered to bind to each other when the affinity (KD) between them is <10⁻⁴ M, <10⁻⁵ M, <10⁻⁶ M, <10⁻⁷ M, <10⁻⁸ M, <10⁻⁹ M, <10⁻¹⁰M, <10⁻¹¹ M, or <10⁻¹² M. One skilled in the art is familiar with how to assess the affinity of two entities (e.g., two proteins).

The term “antibody” encompasses whole antibodies (immunoglobulins having two heavy chains and two light chains), antibody mimetics, and antibody fragments. An “immunoglobulin (Ig)” is a large, Y-shaped protein produced mainly by plasma cells that is used by the immune system to neutralize an exogenous substance (e.g., a pathogens such as bacteria and viruses). Antibodies may be classified as IgA, IgD, IgE, IgG, and IgM. “Antibody fragments” include any antigen binding fragment (i.e., “antigen-binding portion”) or single chain thereof. In some embodiments, an “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. In some embodiments, an antibody is an immunoglobulin (Ig) monomer. An antibody may be a polyclonal antibody or a monoclonal antibody.

In some embodiments, an antibody is a heterotetrameric glycoprotein composed of two identical L chains and two H chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the a and y chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of non-limiting examples of different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6, incorporated herein by reference. In some embodiments, an antibody is an IgG.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, β, ε, γ and μ, respectively. The γ and α classes are further divided into subclasses on the basis of relatively minor differences in CH sequence and function, e.g., humans express the following subclasses: IgG1, IgG2, IgG3, IgG4, IgA1, and IgA2.

The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, Md. (1991), incorporated herein by reference). The constant domains are not involved directly in binding an antibody to an antigen, but exhibit various effector functions, such as participation of the antibody in antibody dependent cellular cytotoxicity (ADCC).

In some embodiments, the antibody is a monoclonal antibody. A “monoclonal antibody” is an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256: 495 (1975), or may be made using recombinant DNA methods in bacterial, eukaryotic animal or plant cells (see, e.g., U.S. Pat. No. 4,816,567). Monoclonal antibodies may also be isolated from phage antibody libraries, e.g., using the techniques described in Clackson et al., Nature, 352: 624-628 (1991) and Marks et al., J. Mol. Biol., 222: 581-597 (1991), incorporated herein by reference.

The monoclonal antibodies described herein encompass “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81: 6851-6855 (1984)). Chimeric antibodies of interest herein include “primatized” antibodies comprising variable domain antigen-binding sequences derived from a non-human primate (e.g. Old World Monkey, Ape etc.), and human constant region sequences.

In some embodiments, the antibody is a polyclonal antibody. A “polyclonal antibody” is a mixture of different antibody molecules which react with more than one immunogenic determinant of an antigen. Polyclonal antibodies may be isolated or purified from mammalian blood, secretions, or other fluids, or from eggs. Polyclonal antibodies may also be recombinant. A recombinant polyclonal antibody is a polyclonal antibody generated by the use of recombinant technologies. Recombinantly generated polyclonal antibodies usually contain a high concentration of different antibody molecules, all or a majority of (e.g., more than 80%, more than 85%, more than 90%, more than 95%, more than 99%, or more) which are displaying a desired binding activity towards an antigen composed of more than one epitope.

In some embodiments, the antibodies are “humanized” for use in human (e.g., as therapeutics). “Humanized” forms of non-human (e.g., rodent) antibodies are chimeric antibodies that contain minimal sequence derived from the non-human antibody. Humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321: 522-525 (1986); Riechmann et al., Nature 332: 323-329 (1988); and Presta, Curr. Op. Struct. Biol. 2: 593-596 (1992).

In some embodiments, the antibody is an “antibody fragment” containing the antigen-binding portion of a full-length ICAM1 antibody. In some embodiments, an antibody is a single domain heavy chain antibody. In some embodiments, an antibody is a single domain light chain antibody. The antigen-binding portion of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (e.g., as described in Ward et al., (1989) Nature 341: 544-546, incorporated herein by reference), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242: 423-426; and Huston et al. (1988) Proc. Natl. Acad. Sci. USA 85: 5879-5883, incorporated herein by reference). Such single chain antibodies are also intended to be encompassed within the term “antigen-binding portion” of an antibody. These antibody fragments are obtained using conventional techniques known to those with skill in the art, and the fragments are screened for utility in the same manner as are full-length antibodies.

In some embodiments, an antibody fragment may be a Fc fragment, a Fv fragment, or a single-change Fv fragment. The Fc fragment comprises the carboxy-terminal portions of both H chains held together by disulfides. The effector functions of antibodies are determined by sequences in the Fc region, which region is also the part recognized by Fc receptors (FcR) found on certain types of cells.

The Fv fragment is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

Single-chain Fv also abbreviated as “sFv” or “scFv” are antibody fragments that comprise the VH and VL antibody domains connected into a single polypeptide chain.

Preferably, the sFv polypeptide further comprises a polypeptide linker between the VH and VL domains which enables the sFv to form the desired structure for antigen binding (e.g., as described in Pluckthun in The Pharmacology of Monoclonal Antibodies, vol. 113, Rosenburg and Moore eds., Springer-Verlag, New York, pp. 269-315 (1994); Borrebaeck 1995, incorporated herein by reference). In some embodiments, an antibody is a dimerized scFV (a diabody), a scFV timer (a triabody), or a scFV tetrameter (a tetrabody).

Antibodies of the present disclosure include antibody mimetics, including affibody molecules. An affibody is a small protein comprising a three-helix bundle that functions as an antigen binding molecule (e.g., an antibody mimetic). Generally, affibodies are approximately 58 amino acids in length and have a molar mass of approximately 6 kDa. Affibody molecules with unique binding properties are acquired by randomization of 13 amino acids located in two alpha-helices involved in the binding activity of the parent protein domain. Specific affibody molecules binding a desired target protein can be isolated from pools (libraries) containing billions of different variants, using methods such as phage display.

In some embodiments, a ICAM1 antibody binds to an epitope that is present in the extracellular portion of an ICAM1. An “extracellular portion” of an ICAM1 refers to the portion of the ICAM1 that is outside of the cytosol and on the surface of the cell (as opposed to the portion that is inside the cytosol. The extracellular portion of an ICAM1 typically comprises

Methods of producing antibodies (e.g., monoclonal antibodies or polyclonal antibodies) are known in the art. For example, a polyclonal antibody may be prepared by immunizing an animal, preferably a mammal, with an allergen of choice followed by the isolation of antibody-producing B-lymphocytes from blood, bone marrow, lymph nodes, or spleen. Alternatively, antibody-producing cells may be isolated from an animal and exposed to an allergen in vitro against which antibodies are to be raised. The antibody-producing cells may then be cultured to obtain a population of antibody-producing cells, optionally after fusion to an immortalized cell line such as a myeloma. In some embodiments, as a starting material B-lymphocytes may be isolated from the tissue of an allergic patient, in order to generate fully human polyclonal antibodies. Antibodies may be produced in mice, rats, pigs (swine), sheep, bovine material, or other animals transgenic for the human immunoglobulin genes, as starting material in order to generate fully human polyclonal antibodies. In some embodiments, mice or other animals transgenic for the human immunoglobulin genes (e.g. as disclosed in U.S. Pat. No. 5,939,598), the animals may be immunized to stimulate the in vivo generation of specific antibodies and antibody producing cells before preparation of the polyclonal antibodies from the animal by extraction of B lymphocytes or purification of polyclonal serum.

Monoclonal antibodies are typically made by cell culture that involves fusing myeloma cells with mouse spleen cells immunized with the desired antigen (i.e., hyrbidoma technology). The mixture of cells is diluted and clones are grown from single parent cells on microtitre wells. The antibodies secreted by the different clones are then assayed for their ability to bind to the antigen (with a test such as ELISA or Antigen Microarray Assay) or immuno-dot blot. The most productive and stable clone is then selected for future use.

### II. Drugs

Drugs suitable for use in the ADCs include agents that are therapeutically active against pancreatic cancer. Non-limiting examples of drugs include chemotherapies. In some instances, a drug is a small molecule. In some embodiments, a drug is a cytotoxic small molecule. In some embodiments, a drug is a cytostatic small molecule.

Non-limiting examples of drugs suitable for use in the ADCs include N2′-Deacetyl-N2′-(3-mercapto-1-oxopropyl)mertansine (DM1), monomethyl auristatin E (MMAE), monomethyl auristatin F (MMAF), and duocarmycin, paclitaxel, everolimus, fluorouracil (5-FU), gemcitabine, gemcitabine hydrochloride, mitomycin C, and derivatives thereof. In some embodiments, the drug is maytansine or an analog thereof. In some embodiments, the drug is DM1. DM1 is a cytotoxic maytansine analog that has been shown to inhibit tubulin polymerization. In some embodiments, the maytansine analog is DM4.

The term “small molecule” refers to molecules, whether naturally-occurring or artificially created (e.g., via chemical synthesis) that have a relatively low molecular weight. Typically, a small molecule is an organic compound (e.g., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible.

Any known chemotherapeutic drugs may be used as the drug in the ADC described herein. Non-limiting exemplary chemotherapetic drugs include: Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, and Vinorelbine.

### III. Linkers

One or more drugs may be conjugated to an ICAM1 antibody using techniques known in the art. In some embodiments, multiple (e.g., e.g., 2, 3, 4, 5, 6, 7, 8, 9, 10, or more) drugs are conjugated to an ICAM1 antibody. The ratio of the ICAM1 antibody and the drug in the ADC may be 1:1 to 1:10 (e.g., 1:1, 1:2, 1:3, 1:4, 1:5, 1:6, 1:7, 1:8, 1:9, or 1:10). In some embodiments, the ratio of the ICAM1 antibody and the drug in the ADC is 1:4.

An ICAM1 antibody may be conjugated to a second entity either directly or via a linker. As used herein, “conjugated” or “attached” means two entities are associated, preferably with sufficient affinity that the therapeutic or diagnostic benefit of the association between the two entities is realized. In some embodiments, a linker conjugates an ICAM1 antibody to a drug in an ADC. The N-terminus or C-terminus of an ICAM1 antibody may be conjugated to a drug. In some embodiments, a linker can be used to conjugate an ICAM1 antibody to an imaging agent. The N-terminus or C-terminus of an ICAM1 antibody may be conjugated to an imaging agent.

In some embodiments, a linker is a cleavable linker. As used herein, a cleavable linker is capable of releasing a conjugated moiety in response to a stimulus. In some embodiments, the stimulus is a physiological stimulus. Non-limiting examples of stimuli include the presence of an enzyme, acidic conditions, basic conditions, or reducing conditions. For example, cleavable linkers include peptide linkers, β-glucuronide linkers, glutathione-sensitive linkers (or disulfide linkers) and pH-sensitive linkers. In some embodiments, a pH-sensitive linker is cleaved at a pH between 5.0 and 6.5 or between a pH of 4.5 and 5.0. In some embodiments, a pH-sensitive linker is not cleaved when the pH is between 7 and 7.5. In some embodiments, a pH-sensitive linker is not cleaved when the pH is between 7.3 and 7.5. In some embodiments, a cleavable linker is a protease-sensitive linker.

Examples of cleavable linkers include N-succinimidyl 4-(2-pyridyldithio)pentanoate (SPP), N-succinimidyl 3-(2-pyridyldithio)butanoate (SPDB), Sulfo-SPDB, valine-citrulline dipeptide (Val-cit), acetyl butyrate, and CL2A. In some embodiments, the cleavable linker is Val-cit. See also, e.g., Donaghy, MAbs. 2016 May-June; 8(4): 659-71.

In some embodiments, a linker is non-cleavable. In some embodiments, a non-cleavable linker is a linker that is not cleaved within systemic circulation in a subject. In some embodiments, a non-cleavable linker is a linker that is resistant to protease cleavage. Non-cleavable linkers include N-succinimidyl 4-(Nmaleimidomethyl)cyclohexane-1-carboxylate (SMCC) and maleimidomethyl cyclohexane-1-carboxylate (MCC). In some embodiments, a non-cleavable linker is a N-succinimidyl 4-(Nmaleimidomethyl)cyclohexane-1-carboxylate (SMCC) linker.

Any of the antibody-drug conjugates may be synthesized using methods known in the art. See, e.g., Yao et al., Int J Mol Sci. 2016 Feb. 2; 17(2). pii: E194.

The ADCs comprising ICAM1 antibody conjugated to a drug are also advantageous to use therapeutically, in part because the drugs (e.g., chemotherapeutic drugs) are toxic and cause severe side effects. By conjugate the drug (e.g., DM1) to the ICAM1 antibody, the toxicity of the ADC may be reduced by at least 20%, at least 30%, at least 40%, at least 50%, at least 60%, at least 70%, at least 80%, at least 90%, at least 99%, compared to the drug in its free from.

### Other ICAMI Antibody Conjugates

ICAM1 antibodies and/or any of the ADCs of the present disclosure may be conjugated to an imaging agent, which may be useful for predicting the therapeutic sensitivity of a subject with pancreatic cancer. For example, imaging agents for computed tomography (CT), positron emission tomography (PET), magnetic resonance imaging (MRI), and endoscopic detection (e.g., endoscopic ultrasound) may be used and can include contrast agents. See, e.g., Bird-Lieberman et al., Nat Med. 2012; 18(2): 315-21; Van den Brande et al., Gut. 2007; 56(4): 509-17, which is each herein incorporated by reference. In some embodiments, the contrast agent is administered as a salt. In some embodiments, the imaging agent is a gadolinium-based MRI contrast agent. For example, an imaging agent may be a gadolinium-diethylenetriamine pentaacetic acid (Gd-DTPA or DTPA-Gd). See, e.g., Can et al., AJR Am J Roentgenol. 1984 August; 143(2): 215-24.

One or more imaging agents may be conjugated to an ICAM1 antibody or an ADC described herein using techniques known in the art. In some embodiments, multiple (e.g., e.g., 2, 3, 4, 5, 6, 7, 8, 9, 10, or more) imaging agents are conjugated to an ICAM1 antibody. The ratio of the ICAM1 antibody or ADC and the imaging agent may be 1:1 to 1:10 (e.g., 1:1, 1:2, 1:3, 1:4, 1:5, 1:6, 1:7, 1:8, 1:9, or 1:10). In some embodiments, the ratio of the ICAM1 antibody or ADC and the imaging agent is 1:4. Any of the linkers disclosed herein may be used to conjugate an imaging agent to an ICAM1 antibody or to an ADC described herein.

An imaging agent may be visualized with a suitable detection method (e.g., by CT, PET, MRI, ultrasound, and/or endoscopic detection).

### Pharmaceutical Compositions and Uses Thereof

Compositions comprising any of the ADCs or other ICAM1 antibody conjugates disclosed herein are encompassed by the present disclosure. In some embodiments, the composition is formulated as a pharmaceutical composition for administration to a subject.

A subject may have, be suspected of having, or be at risk for pancreatic cancer. Pancreatic cancers are classified based on the cell type that starts the tumor. The most common type of pancreatic cancer are pancreatic adenocarcinomas, which are cancers of the exocrine pancreas. In contrast, pancreatic neuroendocrine tumors (NETs), or islet cell tumors, start in neuroendocrine cells.

Pancreatic cancers may also be stratified based on whether or not the cancer has metastasized. A pancreatic cancer may be stage 0 (carcinoma in situ), stage I, stage II (e.g., stage IIA or stage IIB), stage III, or stage (IV). A non-limiting staging method is the TNM system, which evaluates the extent of the tumor (T), the spread of the cancer to nearby lymph nodes (N), and whether the cancer has spread to distant sites (M). The various T, N, and M levels (e.g., Table 1) may then be used to determine the stage of pancreatic cancer (e.g., Table 2). Tables 1-2 show pancreatic tumor classification based on the Eighth Edition of the AJCC/UICC TNM staging system and as described by Cong et al. Sci Rep. 2018 Jul. 10; 8(1): 10383.

TABLE 1
Non-limiting examples of TNM staging definitions

| | |
|---|---|
| T1 | Maximum tumor diameter ≤2 cm |
| T2 | Maximum tumor diameter >2, ≤4 cm |
| T3 | Maximum tumor diameter >4 cm |
| T4 | Tumor involves the celiac axis, common |
| N0 | No regional lymph node metastasis |
| N1 | Metastasis in 1-3 regional lymph nodes |
| N2 | Metastasis in ≥4 regional lymph nodes |
| M0 | No distant metastasis |
| M1 | Distant metastasis |

TABLE 2
Pancreatic Staging Levels

| | T | N | M | T | N | M |
|---|---|---|---|---|---|---|
| IA | T1 | N0 | MO | T1 | N0 | M0 |
| IB | T2 | N0 | M0 | T2 | N0 | M0 |
| IIA | T3 | N0 | M0 | T3 | N0 | M0 |
| IIB | T1-T3 | N1 | M0 | T1-T3 | N1 | M0 |
| III | T4 | any N | M0 | T4 | any N | M0 |
| IV | any T | any N | M1 | any T | Any N | M1 |

In some embodiments, any of the pharmaceutical compositions disclosed herein comprising an imaging agent is administered in an effective amount to a subject to determine the level of ICAM1 in a tumor of a subject with pancreatic cancer (e.g., CT, PET, MRI, and endoscopic detection (e.g., endoscopic ultrasound)). The imaging methods for determining the level of ICAM1 described herein are advantageous compare to conventional methods (e.g., biopsy and analyzing the tissue obtained from the biopsy). The imaging methods (e.g., MRI) is non-invasive, and provides a comprehensive view of the tumor for ICAM1 level, providing more accurate assessment of the tumor for prediction of outcome and/or responsiveness to treatment (e.g., treatment with ICAM1 antibody or ADC comprising ICAM1 antibody).

In some embodiments, the level of ICAM1 is detected in a subject with pancreatic cancer who has been administered a pharmaceutical composition of the present disclosure comprising an ICAM1 antibody and an imaging agent. In some embodiments, the ICAM1 level detected in the tumor of the subject is at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% higher than a control. In some embodiments, the ICAM1 level detected in the tumor of the subject is substantially similar to the control.

In some embodiments, a control is a subject with a tumor having a known level of ICAM1. In some embodiments, a control is the level of ICAM1 in the pancreas of a subject who does not have a tumor. In some embodiments, a control is a subject with a tumor having a low level of ICAM1. In some embodiments, a low level of ICAM1 is not detectable. In some embodiments, a control is a subject with a tumor having a high level of ICAM1. In some embodiments, a high level of ICAM1 is at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% higher than the level of ICAM1 detected in a pancreas of a healthy subject.

In some embodiments, the level of ICAM1 detected in a tumor using a method disclosed herein is indicative of a subject with pancreatic cancer responding to treatment with an of treatment with an ICAM1 antibody or an antibody drug conjugate (ADC) comprising an intercellular adhesion molecule 1 (ICAM1) antibody conjugated to a drug. In some embodiments, a higher level of ICAM1 detected in a tumor as compared to the tumor of a subject with a lower level of ICAM1 is identified as being more responsive to treatment with an ICAM1 antibody or an ADC disclosed herein. In some embodiments, a subject with a higher level of ICAM1 in a tumor as compared to a subject with a lower level of ICAM1 in a tumor is at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% more responsive to treatment with a composition comprising an ICAM1 antibody (e.g., an ICAM1 ADC and/or an ICAM1 antibody not conjugated to a drug) In some embodiments, a method disclosed herein comprises administering an ICAM1 antibody or an ADC antibody disclosed herein after identifying the subject as being responsive.

In some embodiments, the level of ICAM1 detected in a tumor using a method disclosed herein is indicative of the stage of pancreatic cancer. In some embodiments, the level of ICAM1 detected in a tumor is indicative of stage 0, stage I, stage II, stage III, or stage IV.

Without being bound by a particular theory, in some embodiments, administration of an ICAM1 antibody conjugated to an imaging agent or an ICAM1 ADC conjugated to an imaging agent may serve a dual purpose of visualizing a pancreatic tumor and treating the tumor.

In some embodiments, administration of an ICAM1 antibody and/or an ADC comprising an ICAM1 antibody or a pharmaceutical composition thereof inhibits the growth of a tumor. In some embodiments, administration of an ICAM1 antibody and/or an ADC comprising an ICAM1 antibody or a pharmaceutical composition thereof results in regression of a tumor. In some embodiments, administration of an ICAM1 antibody and/or an ADC comprising an ICAM1 antibody or a pharmaceutical composition thereof decreases the size of a tumor by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% as compared to a control. In some embodiments, the control is a subject who has not been treated with a composition that comprises an ICAM1 antibody.

In some embodiments, administration of an ICAM1 antibody and/or an ADC comprising an ICAM1 antibody or a pharmaceutical composition thereof disclosed herein decreases proliferation by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% higher than a control. In some embodiments, proliferation is measured using Ki67 staining. In some embodiments, the control is a subject who has not been treated with a composition that comprises an ICAM1 antibody.

In some embodiments, administration of an ICAM1 antibody and/or an ADC comprising an ICAM1 antibody or a pharmaceutical composition thereof disclosed herein decreases metastasis of a tumor by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% as compared to a control. In some embodiments, the control is a subject who has not been treated with a composition that comprises an ICAM1 antibody.

In some embodiments, administration of an ICAM1 antibody and/or an ADC comprising an ICAM1 antibody or a pharmaceutical composition thereof disclosed herein does not decrease the viability of healthy cells. In some embodiments, administration of an ADC or a pharmaceutical composition comprising an ADC disclosed herein allows for the effective amount (e.g., concentration) of a drug to be lower than if the drug was not conjugated to an ICAM1 antibody. In some embodiments, the effective amount of a drug is lowered by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% as compared to administration of the drug alone.

In some embodiments, the pharmaceutical composition further comprises a pharmaceutically acceptable carrier. “Pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier” may be a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the pharmaceutical compositions also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The pharmaceutical compositions may conveniently be presented in unit dosage form and may be prepared by any of the methods well-known in the art of pharmacy. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

The formulation of the pharmaceutical composition may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol.

Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

In some embodiments, the pharmaceutical compositions used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The pharmaceutical composition ordinarily will be stored in lyophilized form or as an aqueous solution if it is highly stable to thermal and oxidative denaturation. The pH of the preparations typically will be about from 6 to 8, although higher or lower pH values can also be appropriate in certain instances.

“A therapeutically effective amount” or “effective amount” as used herein refers to the amount of each therapeutic agent (e.g., therapeutic agents for treating any of the brain disease described herein) of the present disclosure required to confer therapeutic effect on the subject, either alone or in combination with one or more other therapeutic agents. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons.

Empirical considerations, such as the half-life, generally will contribute to the determination of the dosage. For example, therapeutic agents that are compatible with the human immune system, such as polypeptides comprising regions from humanized antibodies or fully human antibodies, may be used to prolong half-life of the polypeptide and to prevent the polypeptide being attacked by the host's immune system. Frequency of administration may be determined and adjusted over the course of therapy, and is generally, but not necessarily, based on treatment and/or suppression and/or amelioration and/or delay of a disease. Alternatively, sustained continuous release formulations of a polypeptide may be appropriate. Various formulations and devices for achieving sustained release are known in the art.

In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the anti-cancer agent used) can vary over time.

In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the anti-cancer agent (such as the half-life of the anti-cancer agent, and other considerations well known in the art).

For the purpose of the present disclosure, the appropriate dosage of a therapeutic agent as described herein will depend on the specific agent (or compositions thereof) employed, the formulation and route of administration, the type and severity of the disease, whether the anti-cancer agent is administered for preventive or therapeutic purposes, previous therapy, the subject's clinical history and response to the antagonist, and the discretion of the attending physician. Typically the clinician will administer an anti-cancer agent until a dosage is reached that achieves the desired result. Administration of one or more anti-cancer agents can be continuous or intermittent, depending, for example, upon the recipient's physiological condition, whether the purpose of the administration is therapeutic or prophylactic, and other factors known to skilled practitioners. The administration of an anti-cancer agent may be essentially continuous over a preselected period of time or may be in a series of spaced dose, e.g., either before, during, or after developing a disease.

As used herein, the term “treating” refers to the application or administration of an anti-cancer agent to a subject in need thereof. “A subject in need thereof”, refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease.

A “subject” to which administration is contemplated refers to a human (i.e., male or female of any age group, e.g., pediatric subject (e.g., infant, child, or adolescent) or adult subject (e.g., young adult, middle—aged adult, or senior adult)) or non-human animal. In some embodiments, the non—human animal is a mammal (e.g., rodent (e.g., mouse or rat), primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). The non-human animal may be a male or female at any stage of development. The non-human animal may be a transgenic animal or genetically engineered animal.

In some embodiments, the subject is a companion animal (a pet). “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include: rodents (e.g., rats, mice, guinea pigs, and hamsters), rabbits, or non-human primates.

Alleviating a disease (e.g., cancer) includes delaying the development or progression of the disease, or reducing disease severity. Alleviating the disease does not necessarily require curative results. As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease includes initial onset and/or recurrence.

Conventional methods, known to those of ordinary skill in the art of medicine, can be used to administer the pharmaceutical composition the subject, depending upon the type of disease to be treated or the site of the disease. The pharmaceutical composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In some embodiments, the pharmaceutical composition is administered via intravenous injection or infusion. In addition, it can be administered to the subject via injectable depot routes of administration such as using 1-, 3-, or 6-month depot injectable or biodegradable materials and methods. In some embodiments, the pharmaceutical composition is administered via injection. In some embodiments, injection is intravenous injection or intratumoral injection.

### EXAMPLES

### Introduction

To date, pancreatic cancer (PC) remains among the most lethal diseases that accounts for 56,770 people death in the United States within 2019, representing 7% of all cancer mortality. The prognosis for PC patients is strikingly poor with a 5 year survival less than 8% despite of the recent intensified studies of immunotherapy and nanomedicine therapy. For instance, immune checkpoint blockade therapies including cytotoxic T-lymphocyte-associated antigen 4 (CTLA4) inhibitor or Programmed death-ligand 1 (PD-L1) antibody have yet to show enough clinical efficacy in treating PC patients to date. Innovative nanomedicine formulations (e.g., EphA2-targeted liposomal docetaxel) also failed to bring clinical benefits in treating advanced PC. These undesirable results are largely due to the immunosuppressive tumor microenvironment (TME) of PC tumors, which is characterized by desmoplastic stroma and poor vascularization. Such TME creates physical barriers that prevent T-cells or nanomedicines efficiently infiltrating tumors and directly interacting with PC cells, leading to unfavorable efficacies. It highlights a critical need to develop novel targeted therapeutics that can better infiltrated PC tumors while maintaining potent tumor-specific efficacy.

Antibody-drug conjugates (ADCs) are a rapidly growing class of immunotherapeutics that have shown promising clinical efficacy against several types of cancers including aggressive solid tumors like breast cancer, which respond poorly to T-cell immunotherapy. Unlike conventional chemotherapeutics, ADCs utilizes chemical linkers to conjugate cytotoxic drugs to tumor-homing antibodies, which are capable of selectively homing tumors while sparing normal tissues via recognizing tumor surface antigens, subsequently internalizing and delivering cytotoxic drugs into targeted tumor cells. Compared to T-cell immunotherapy (e.g., chimeric antigen receptor-T cell (CAR-T) or immune checkpoint blockade) or nanomedicines (e.g., liposomes or exosomes), ADC features a superior tumor tissue penetration due to its ultrasmall size (<10 nm), which is ˜1,000 fold smaller than the size of a T-cell, creating an attractive opportunity to increase drug delivery into stroma dense PC tumors.

However, a major hurdle in developing PC-targeted ADC is identifying suitable immunotherapeutic targets that effectively distinguish PC and normal tissues. To meet the safety and efficacy criteria for optimal ADC, such targets need to be abundantly presented on the cell surface of PC tumors with undetectable levels in normal tissues, making it assessable to the tumor-homing antibody of ADCs. Meanwhile, it is also required to facilitate a rapid and robust cell internalization of cytotoxic payloads conjugated on ADCs. Though several PC-targeted ADCs have been developed utilizing conventional PC targets (e.g., EGFR, EpHA2, and Mesothelin), there still lacks a systematic and quantitative comparison of established PC targets and other candidates at their cell surface protein levels. The present disclosure shows that performing such unbiased and quantitative screening of cell surface proteins leads to the discovery of more optimal PC immunotherapeutic targets and promoting the development of PC-targeted ADCs.

ICAM1, also called CD54, is a transmembrane glycoprotein of immunoglobulin superfamily, which is aberrantly overexpressed in multiple types of cancers (e.g., triple negative breast cancer) and is frequently associated with an aggressive phenotype and worse prognosis. In PC, ICAM1 is directly induced on pancreatic acinar cells by KRAS^{G12D} mutation, the most common oncogenic mutation in 70-95% PC patients, and drives the formation of pancreatic neoplastic lesions, leading to PC tumor initiation. The present disclosure describes the identification and application of ICAM1 as a potential PC immunotherapeutic target based on an unbiased and quantitative screening algorithm. As such, ICAM1 ADC that induces potent and durable PC tumor regression in vivo was developed. To develop a precision medicine, a non-invasive MRI approach to identify ICAM1-expressing tumors suitable for ICAM1-targeting immunotherapy was designed.

### Results and Discussion

### ICAMI is a Rationally Identified Cell Surface Protein Target for Human PC

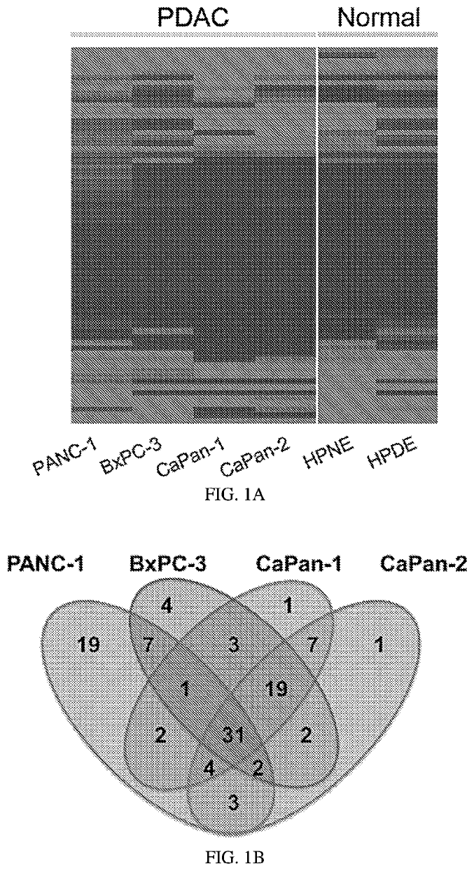

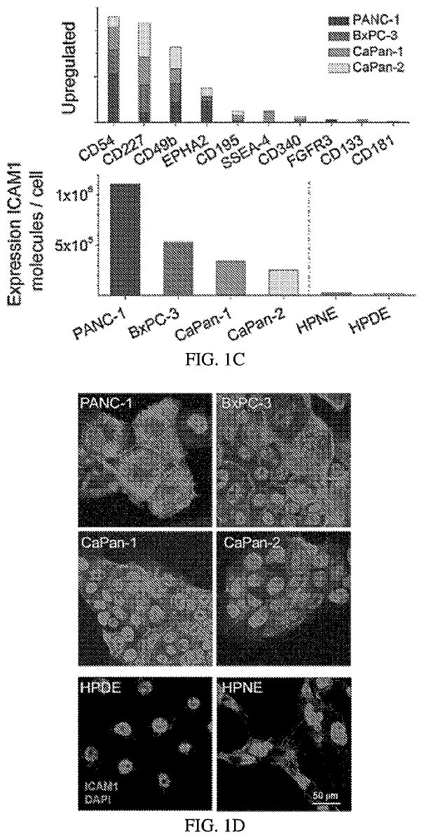

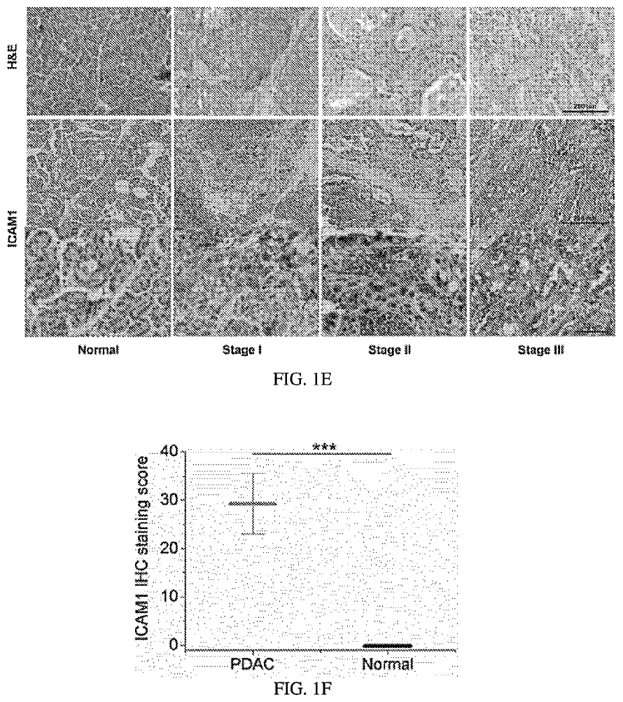

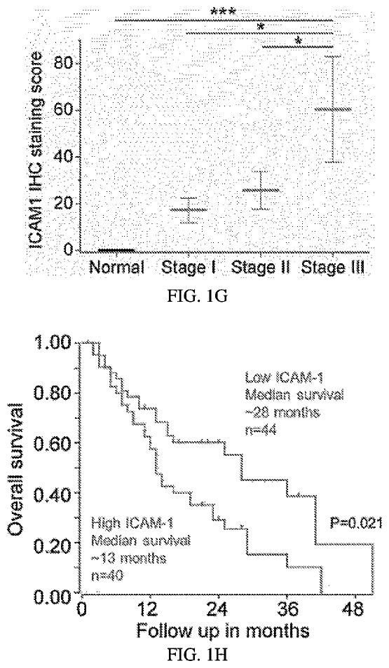

To identify suitable protein targets distinguishing malignant PC tumors from normal tissues, a rationally-designed cell surface protein target discovery algorithm was developed (FIG. 1A). First, an unbiased and quantitative screening of a panel of 72 cancer-related surface antigens in four established human PC cell lines (PANC-1, BxPC-3, Capan-1, and Capan-2) was performed in comparison with two normal human pancreatic duct epithelial cells (HPDE and HPNE) as normal controls (FIG. 1B). Of the 68 screened targets, 31 candidates were found to be commonly overexpressed in all four PC cell lines and were selected for further evaluation. By comparing their levels in human PC cells and normal pancreatic cells, ICAM1 emerged as the most overexpressed PC targets among the top 10 candidates with almost no expression in non-neoplastic HPNE and HPDE cells (FIG. 1C). The cell surface density of ICAM1 ranges from 3×10⁵ to 1×10⁶ molecules/cell on four PC cell lines, significantly higher than those of established PC targets (e.g., EGFR, MUC1, or EphA2). Additionally, ICAM1 is ubiquitously overexpressed across all four tested human PC cell lines, suggesting a broad target population in PC patients. The overexpression of ICAM1 in human PC cells was further confirmed using immunofluorescent (IF) staining. ICAM1 was predominantly expressed on the plasma membranes of four PC cell lines (PANC-1, BxPC-3, Capan-1 and Capan-2) but absent in normal HNPE and HPNE cells (FIG. 1D). This strong cell surface expression of ICAM1 on human PC cells makes it readily assessable for ICAM1-targeting immunotherapeutics (e.g., ADCs or CAR-T cells).

To investigate whether high ICAM1 expression is a clinically relevant finding in human PC, an immunohistochemical (IHC) staining of ICAM1 was conducted in 80 human PC tumor tissues and 20 normal pancreas tissues. In FIG. 1E and FIG. 1F, ICAM1 was consistently overexpressed on plasma membrane and in cytoplasm of PC cells from tumor tissues at different disease stages, and ICAM1 is completely absent in the normal human pancreas tissues. The extent of staining and the pathological scores of ICAM1 showed that ICAM1 level was positively correlated to the disease TNM stages (FIG. 1G). The on-target, off-tumor sites for ICAM1-targeting immunotherapeutics in normal tissues were also evaluated. The protein levels of ICAM1 were examined in a comprehensive cohort of 45 normal human organs by querying Human Protein Atlas database (proteinatlas.org). It was found that ICAM1 expression was absent in most normal tissues by IHC analysis, and only 4% ( 2/45, lung and kidney) of normal tissues show high positive staining of ICAM1, respectively. This suggested that lung and kidney are potential on-target, off-tumor sites for ICAM1-targeting immunotherapeutics. Moreover, previous work has shown ICAM1-targeting T-cell immunotherapy does not induce any acute or delayed toxicity in both male and female mice of advanced thyroid cancer model.

The impact of ICAM1 overexpression on clinical outcomes of PC patients was investigated by querying the R2: Genomics Analysis and Visualization Platform database (hgserver1.amc.nl/), Datasheet: Mixed Pancreas Tumor-Zhang). The overall survival of PC patients with high ICAM1 expression was significantly worse than those with low ICAM1 expression (FIG. 1H, P=0.021, log-rank test), suggesting that ICAM1 may serve as a clinical biomarker of poor prognosis in PC patients.

### ICAMI Antibody Recognizes and Targets PC Tumor In Vivo

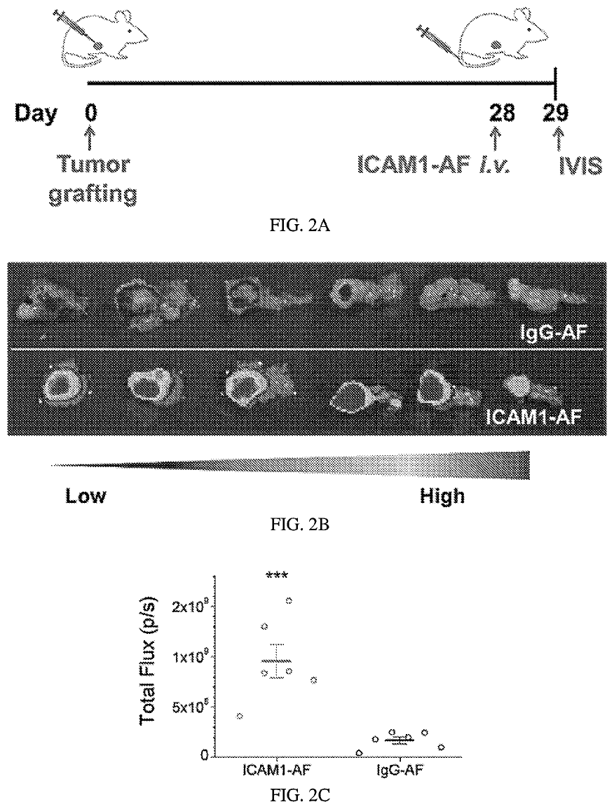

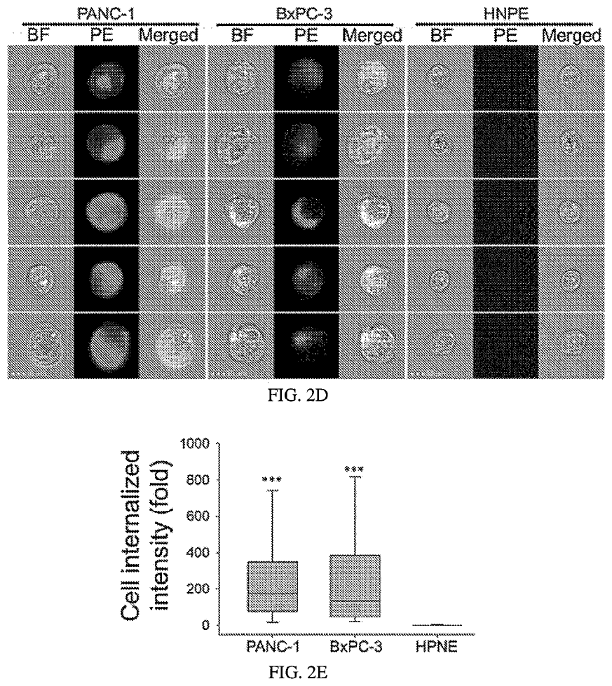

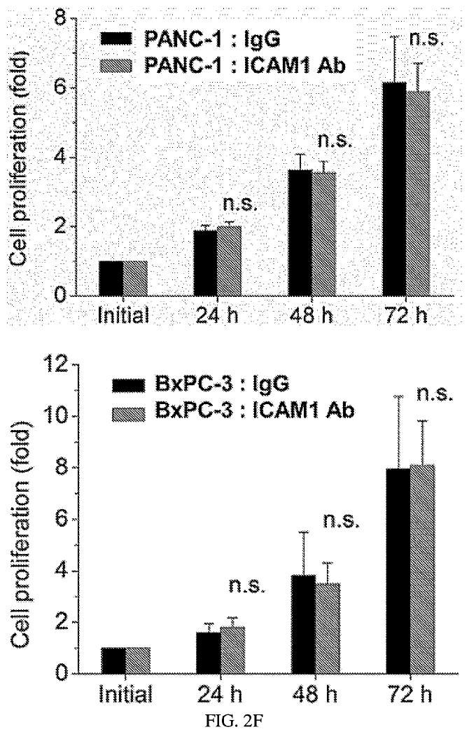

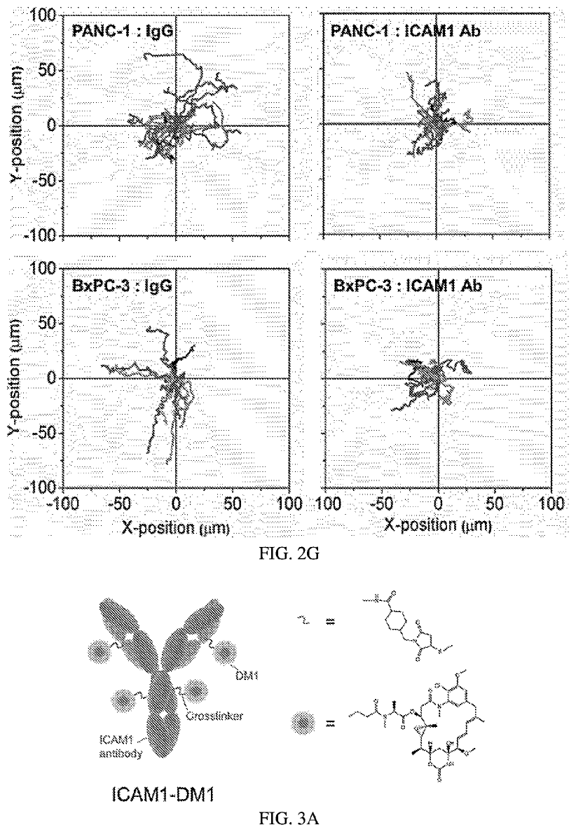

To assess ICAM1 as a potential immunotherapeutic target, the in vivo tumor-specificity of ICAM1 antibody was first determined in an orthotopic PC tumor model (FIG. 2A). ICAM1 monoclonal antibodies were fluorescently labeled with AF-647, a red fluorescent dye, and intravenously injected into PANC-1 tumor-bearing mice. AF-647 labeled IgG (IgG-AF) was used as a non-targeting control. Due to the fact that in vivo fluorescent signal was interfered by the intraperitoneal location of orthotopic PC tumors and abdominal skin absorption, the animals were euthanized at 24 hours-post injection and PC tumors and their surrounding pancreatic tissues were excised. Then, ex vivo imaging was performed to determine the tumoral accumulation of ICAM1-AF antibodies. As observed in FIG. 2B, ICAM1 antibody selectively recognized and targeted orthotopic PC tumors with high affinity compared with non-targeting IgG controls. Normal pancreatic tissues adjacent to PC tumors were not targeted by ICAM1 antibody, further confirming its PC tumor-specificity. Quantified fluorescent signals (FIG. 2C) confirmed that the tumoral accumulation of ICAM1 antibody was approximately 6-fold higher than that of non-targeting IgG-AF after one single dose of tail-vein administration. These in vivo findings strongly support the development of ICAM1 antibody-based immunotherapeutics for PC-targeted therapy.

Given that cell entry activity is a critical factor in ADC design, cell internalization of ICAM1 antibodies in human PC cells was investigated using an imaging flow cytometry assay. As shown in FIG. 2D, phycoerythrin (PE)-conjugated ICAM1 antibodies were robustly internalized by both PANC-1 and BxPC-3 cells via ICAM1 antigen-mediated endocytosis, whereas almost no PE-ICAM1 antibodies were internalized by normal HPNE cells due to the significant lack of ICAM1 antigen expression. The internalized amount of PE-ICAM1 antibody by human PC cells was quantified as approximately 300-fold higher than that of HPNE cells (FIG. 2E).

Next, the therapeutic consequences of blocking ICAM1 signaling cascades in human PC cells was investigated using its neutralizing antibody. In FIG. 2F, treatment with ICAM1 neutralizing antibody (2 μg/mL) did not obviously alter PANC-1 or BxPC-3 cell proliferation. However, ICAM1 neutralizing antibodies did potently inhibit PANC-1 and BxPC-3 cell migration in comparison with IgG controls, which reduced cell migration of PANC-1 and BxPC-3 cells by 39% and 44%, respectively (FIG. 2G). Correlatively, ICAM1 neutralizing antibodies have also been reported to potently inhibit PC tumor initiation in vivo. These findings indicate that ICAM1 can serve as a PC tumor-homing target. They also indicate that that targeting ICAM1 signaling cascades can also hinder disease progression.

### Rational Design of ICAM1 Antibody-Drug Conjugates

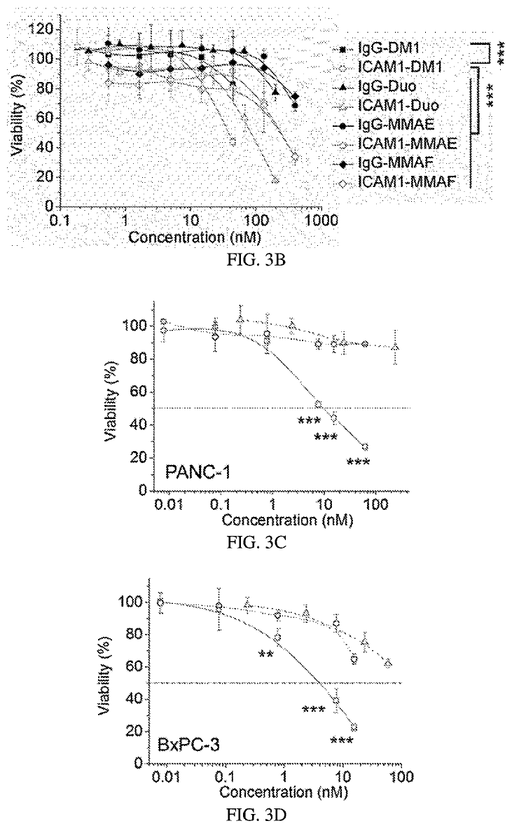

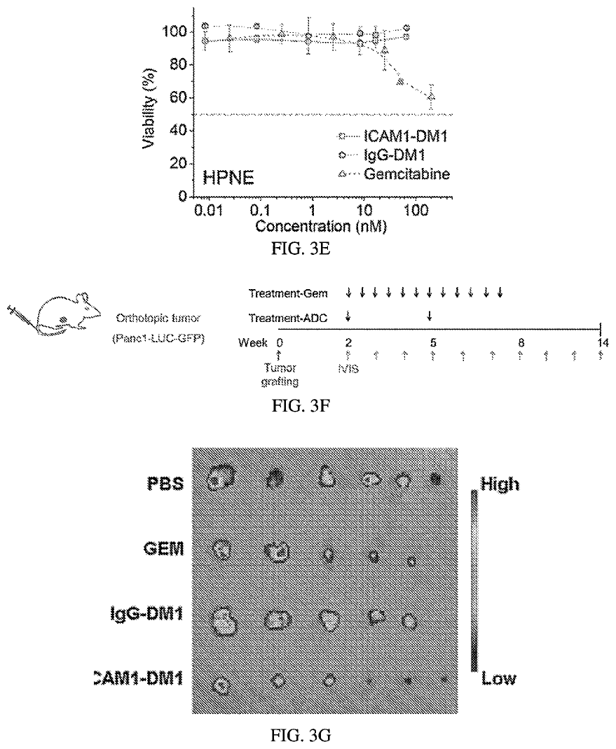

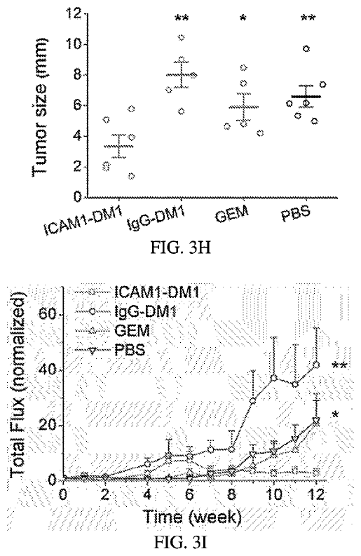

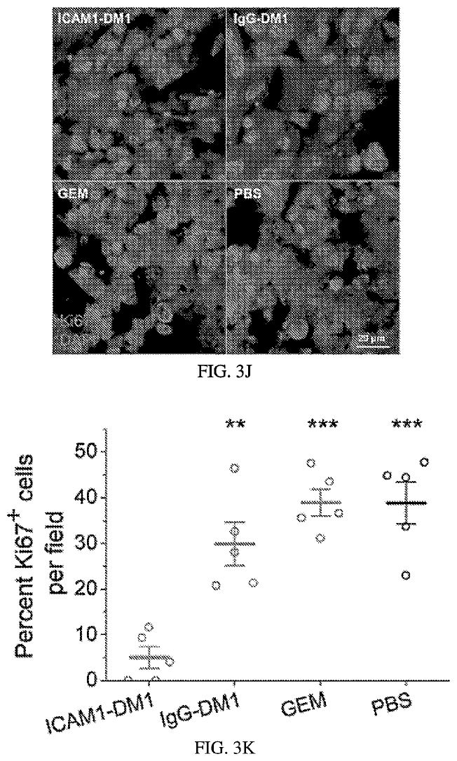

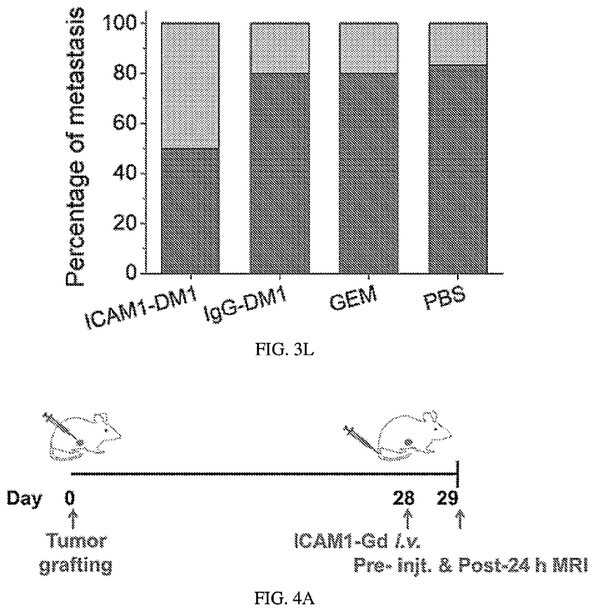

To translate ICAM1 target into PC therapy, a rationally-designed ICAM1 ADC was designed as an immunotherapeutic for PC-targeted therapy (FIG. 3A). Given that chemical linker and cytotoxic payload substantially affect the efficacy of ADC, the first step was to select the optimal ADC formulation for PC treatment using an unbiased and quantitative screening approach. A series of ICAM1 ADCs was engineered using four clinically-tested ADC linkers and cytotoxic payloads (SMCC-DM1, Vc-MMAE, Mc-MMAF, Duocarmycin) at equivalent drug-to-antibody ratio (DAR) of 1 and compared their cytotoxicity against human PC cells, in comparison with non-targeting IgG ADC controls. As shown in FIG. 3B, ICAM1-SMCC-DM1 showed the lowest IC50 (38.1 nM) among four tested ADC formulations (other IC50: 83.9-240.4 nM) in treating PANC-1 cells. The IC50 of ICAM1-SMCC-DM1 is over 2,000-fold lower than Gem (89.1 μM), the first-line chemotherapeutic for PDAC therapy. SMCC-DM1 is a clinically validated ADC formulation consisting of a non-cleavable chemical linker and a potent microtubule inhibitor, Mertansine (DM1). Thus, SMCC-DM1 was selected as the optimized ADC formulation and subsequently synthesized ICAM1-SMCC-DM1 (ICAM1-DM1) as the optimized ICAM1 ADC for PC-targeted therapy. IgG-SMCC-DM1 (IgG-DM1) was also prepared under the same experimental conditions as a non-targeting control. The drug-to-antibody ratios (DARs) for ICAM1-DM1 and IgG-DM1 were controlled by the input amounts of DM1 and antibodies and achieved 3.4 for ICAM1-DM1 and 3.2 for IgG-DM1 as determined using an UV/VIS spectroscopy assay.

### ICAM1-DM1 Selectively Ablates PC Cells In Vitro and In Vivo

The PC-specific cytotoxicity of ICAM1-DM1 was determined in two human PC cells (PANC-1 and BxPC-3) and normal HPNE cells (FIGS. 3C-3E). First-line chemodrug GEM and non-targeting IgG-DM1 were used as controls. As observed in FIG. 3D and FIG. 3E, ICAM1-DM1 showed potent cytotoxicity against PANC-1 and BxPC-3 cells. The IC50 of ICAM1-DM1 is determined as 9.8 nM for PANC-1 and 4.0 nM for BxPC-3, significantly lower than those of GEM and IgG-DM1 (30 nM-88 μm). Moreover, ICAM1-DM1 shows no cytotoxicity in normal HPNE cells due to their lack of expression of ICAM1 (FIG. 3E). These in vitro results strongly support to evaluate anti-tumor activity of ICAM1-DM1 in the in vivo settings of PC models.

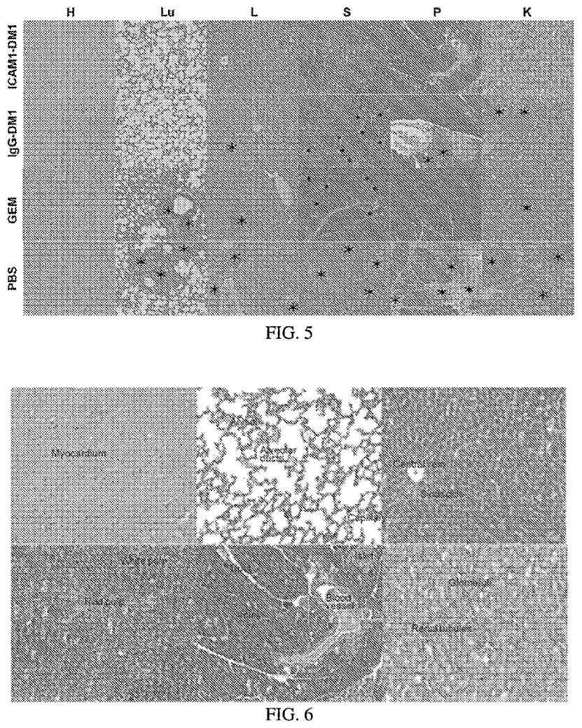

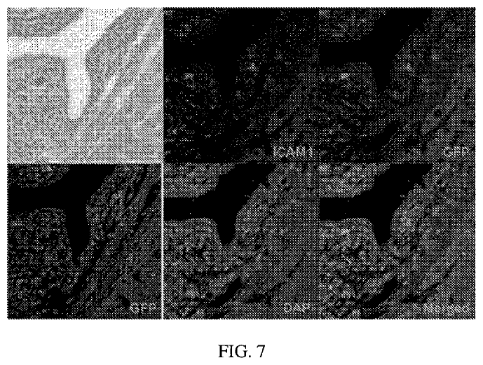

The anti-tumor activity of ICAM1-DM1 was examined in suppressing orthotopic PC tumor growth in vivo (FIG. 3F). ICAM1-DM1 or IgG-DM1 (non-targeting control) were intravenously administered in PANC-1-Luc tumor-bearing mice at 15 mg/kg every 3 weeks. In comparison, GEM was weekly intravenously administered at a dose of 5 mg/kg due to its short circulation half-life (0.28 hr). After two ADC injections, ICAM1-DM1-treated group exhibited a potent and durable tumor regression compared to other groups (FIGS. 3G-3H). The quantified tumor mass showed that ICAM1-DM1 significantly reduced PC tumor growth by 49% in comparison with PBS (sham) group (FIG. 3I). The mechanism of ICAM1-DM1 induced toxicity was further examined by measuring cell proliferation marker Ki67 expression in PC tumor tissues. As observed in FIG. 3K and FIG. 3L, Ki67-positive cell population in ICAM1-DM1-treated group was significantly reduced compared with other groups, contributing to the potent and persistent tumor suppression. This potent anti-tumor activity of ICAM1-DM1 also effectively inhibited spontaneous PC metastasis to normal organs including lung, liver, and spleen (FIG. 3M and FIG. 5). There was no evidence of histopathological damage to the normal vital organs collected from the ICAM1-DM1 treated group.

### Non-Invasively Evaluating Tumoral ICAMI Expression by MRI

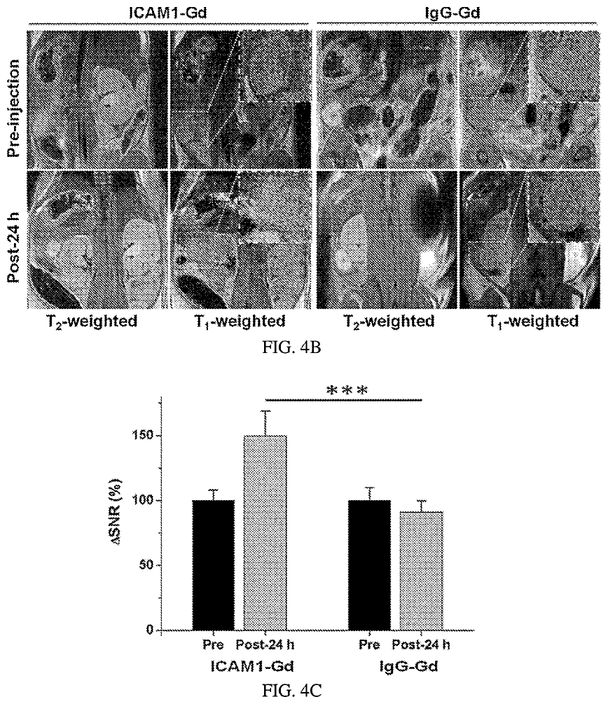

To build a precision medicine, a MRI-based molecular imaging approach was developed for non-invasively and rapidly identifying PC patients that may benefit from ICAM1-targeting immunotherapy. In clinic practice, a needle biopsy is commonly adopted prior to targeted therapy to examine the adequacy of target expression in tumor tissues, but this approach is limited by its invasiveness and the lack of accuracy (<50%) due to the intratumoral complexity and heterogeneity. To overcome these obstacles, an ICAM1-targeting MRI probe was developed and used to map the tumoral ICAM1 expression in an orthotopic PC model using MRI (FIG. 4A). The ICAM1-targeting MRI probe was first engineered by covalently conjugating ICAM1 antibody with DTPA-Gd, a clinically-used MRI contrast agent. IgG-Gd was prepared as a non-targeting control. Then ICAM1-Gd or IgG-Gd was intravenously administered into ICAM1-expressing PANC-1 tumor-bearing mice at a dosage of 5 mg/kg mouse weight. At pre- and 24 hour-post injection of MRI probes, in vivo MRI was performed on PC tumor-bearing mice with a set of MRI sequences, including T1, T2-weighted spin echo imaging. In FIG. 4B, high-resolution T2-weighted MRI images were analyzed to locate PC tumors (yellow circle) in peritoneal cavity. Once the PC tumor was located, T1-weighted MRI images were used to quantitatively measure MRI signal changes in the area of PC tumor (yellow circle) as a function of intratumoral accumulation of gadolinium from administered MRI probes. In FIG. 4C, the tumoral T1 MRI signal increased ˜50% in ICAM1-Gd group, while no MRI signal changes were observed in non-targeting IgG-Gd group (n=3/group). These MRI signal changes are positively correlated with the level of antigen expression on targeted tumors, which can be used to identify ICAM1-positive patients that may benefits from ICAM1-targeting immunotherapy.

In summary, the present disclosure provides experimental evidence that ICAM1 is a suitable ADC target for human PC. The utility of this target can be extended to developing other immunotherapeutics including CAR T cells or bi-specific antibodies directed toward ICAM1.

### Materials and Methods

### Materials

Purified anti-human CD54 Antibody (Clone: HCD54), phycoerythrin (PE)-conjugated mouse anti-human ICAM-1 antibody (PE-ICAM1) and PE conjugated mouse IgG isotype (PE-IgG) were purchased from BioLegend (San Diego, Calif., USA). ADC prescreening G-DM1, G-MMAE, G-MMAF, G-Duoca were purchased from Levena Biopharma (San Diego, Calif.). SMCC-DM1 was purchased from Medkoo (Morrisville, N.C., USA). Zeba™ Spin Desalting Columns, (7K MWCO), Alexa Fluor 647 NHS ester, the Lab-Tek II Chamber Slide System, ProLong Gold Antifade Mountant was obtained from Thermo Fisher Scientific. Gemcitabine hydrochloride (GEM), Gadolinium(III) chloride hexahydrate (GdCl3.6H2O), diethylenetriaminepentaacetic dianhydride (DTPAA), sodium bicarbonate, sodium citrate tribasic dihydrate were purchased from Sigma-Aldrich (St. Louis, Mo.). Dulbecco's PBS, DAPI, Quant-iT RNA Assay Kit, 0.25% trypsin/2.6 mM EDTA solution, Gibco DMEM, Gibco DMEM/F12(1:1), Roswell Park Memorial Institute (RPMI)-1640 medium, and McCoy's 5A medium were purchased from Invitrogen (Carlsbad, Calif.). MEGM Mammary Epithelial Cell Growth Medium was purchased from Lonza (Basel, Switzerland). Quantum Simply Cellular microbeads were purchased from Bangs Laboratory (Fishers, Ind.). The Dojindo cell counting kit CCK-8 was purchased from Dojindo Molecular Technologies (Rockville, Md., USA). Human pancreatic cancer tissue and normal tissue arrays (PA1002a) were purchased from US Biomax (Derwood, Md.).

### Cell Culture

PANC-1, BxPC-3, Capan-1, Capan-2 and HPNE cells were purchased from ATCC (Manassas, VA). HPDE cells were purchased from Kerafast (Boston, MA). PANC-1, Dulbecco's Modified Eagle's Medium with 10% FBS, BxPC-3, RPMI-1640 Medium with 10% FBS; Capan-1, Iscove's Modified Dulbecco's Medium with 20% FBS, Capan-2, Modified McCoy's 5a Medium with 10% FBS; HPNE, 75% DMEM without glucose with additional 2 mM L-glutamine and 1.5 g/L sodium bicarbonate, 25% Medium M3 Base, FBS 5%, 10 ng/ml human recombinant EGF, 5.5 mM D-glucose (1 g/L), 750 ng/ml puromycin; HPDE, Keratinocyte Basal Medium+supplied supplements (Lonza, Clonetics KBM, Cat#CC-3111). All cells were maintained at 37 ° C. in a humidified incubator with 5% (vol/vol) CO₂.

### Quantification of ICAM-1 Surface Expression

Pancreatic cancer cell ICAM1 surface protein expression was evaluated by a BD FACSCalibur flow cytometer (BD Biosciences) as described previously. Quantification of the ICAM-1 density on the cell surface was determined with reference to Quantum Simply Cellular microbeads, using the protocol as provided by the manufacturer. 10⁶ cells were collected and rinsed twice through suspension-spin cycles. Cells were blocked by 1% BSA in PBS for 30 min in an ice bath. After BSA blockage, cells were incubated with phycoerythrin (PE) conjugated ICAM1 antibody for 1 hour at room temperature. Cells were rinsed with 1% BSA in PBS three times, resuspended in PBS, and evaluated by flow cytometry.

### Immunohistological Staining

Immunohistochemical studies were conducted on paraffin-embedded human PDAC and normal tissue microarrays (PA1002a, US Biomax). Forty cases of human PDAC tissue and ten cases of human normal tissue microarray samples were evaluated for ICAM-1 expression as described previously. The individual tissue cores in the microarrays were scored by a surgical pathologist, with no knowledge of sample identity. Immunostains were scored by calculating H-scores in which the percent of cells staining strong (3+), moderate (2+), and weak (1+) were multiplied according to the formula: H-score=3×(% of cells staining 3+)+2×(% of cells staining 2+)+1×(% of cells staining 1+). Photomicrographs were taken on an Olympus BX41 microscope by using an Olympus Q-Color5 digital camera (Olympus America Inc.).

### In Vitro Binding and Internalization of ICAM-1 Ab

The in vitro specific binding of ICAM1 antibody to the human pancreatic cancer cell lines was assessed using PE-ICAM1 antibody. IgG was used as the control. Cells were seeded in 8-well chamber slides at a density of 5×10³ cells/well. After recovering for 24 hours, the full media was replaced with that containing PE-ICAM1 antibody, with 1% FBS. Cells were incubated with the PE-ICAM1-containing media at 37° C. for an additional 4 hours. The cell monolayer was then rinsed with cold phosphate-buffered saline (PBS) and fixed with 4% paraformaldehyde in the PBS solution. Cell nuclei were counterstained with 4′,6′-diamidino-2-phenylindole hydrochloride (DAPI) using ProLong Gold Antifade Mountant. Fluorescence images were acquired and analyzed using a Zeiss LSM 880 confocal microscope (Oberkochen, Germany).

In the imaging flow cytometry studies, cells were seeded in 6-well chamber slides at a density of 1×10⁶ cells/well. After cell recovery for 24 hours, the full media was replaced with that containing PE-ICAM1 antibody, with 1% FBS. Cells were incubated with the PE-ICAM1-containing media at 37° C. for 1 hour. The cell monolayer was then collected and rinsed with cold PBS twice, resuspended and evaluated using an Amnis imagestreamX Mark II imaging flow cytrometry (Luminex, Austin, Tex., USA).

### Therapeutic Effect of ICAM-1 Ab

The in vitro therapeutic effect of ICAM1 antibody to the human pancreatic cancer cell lines was assessed using quantitative phase imaging. IgG was used as the control. Cells were seeded in a 6-well plate at a density of 5×10⁴ cells/well. After recovering for 24 hours, the full media was replaced with that containing ICAM1 antibody or IgG at a dosage of 2 μg/mL. Cells were incubated with the ICAM1- or IgG-containing media at 37° C. for 24 hours. After that, the plate was placed under a quantitative phase imaging microscope (Holomonitor M4, Phase Holographic Imaging Phi AB, Lund, Sweden) setting in an incubator and imaged for an additional 24 hours with a 5 min interval. Cell motion, morphology and proliferation were then analyzed using Hstutio4.

### Preparation and Characterization of ADCs Ab-Gd

IgG-DM1, ICAM1-DM1 were prepared by mix IgG or ICAM1 Ab (2.4 mg, 16 nmol) with SMCC-DM1 (1.2 mg, 1.1 umol) in a phosphate buffer (pH 7.2), rotating at room temperature for 1 h. Free SMCC-DM1 was removed by Ultra-4 Centrifugal Filter (30K MWCO). IgG-DM1, ICAM1-DM1 were washed with PBS (pH 7.4) for several times and redispersed in PBS.

The ADCs were characterized by UV/Vis spectroscopy and antibody drug ratios were calculated according to the equation:

ADR=C_{drug}/C_{Ab}=(A²⁸⁰ε_{λ(D)mAB}−A_{λ(D)}ε_{280mAb})(A₂₈₀ε_{λ(D)drug}−A_{λ(D)}ε_{280drug})/[(ε_{280drug}ε_{λ(D)mAB}−ε_{λ(D)drug}ε_{280mAb})(ε_{280mAb}ε_{λ(D)drug}−ε_{λ(D)}m_{Ab}ε_{280drug})]

IgG-DTPA-Gd, ICAM1-DTPA-Gd were prepared as reported previously. DTPAA (0.4 mg, 1.1 μmol) was slowly added to IgG or ICAM1 Ab (0.2 mg, 1.3 nmol) in NaHCO3 buffer (pH 9.0, 0.1 M), and the mixture was rotated at room temperature overnight. The DTPA-conjugated IgG or ICAM1 Ab was purified by Ultra-4 Centrifugal Filter (30K MWCO) and redispersed in citrate buffer (pH 6.5, 0.1 M). Then GdCl3 (0.1 mg, 0.27 μmol) in 0.1 M citrate buffer (pH 6.5) was mixed with DTPA-conjugated IgG or ICAM1 Ab for 24 h rotated at room temperature. The free Gd³⁺ was removed by Ultra-4 Centrifugal Filter (30K MWCO), and IgG-or ICAM1-DTPA-Gd was redispersed in PBS for subsequent use. The Ab-Gd were characterized by liquid chromatography electrospray ionization mass spectrometry LC-MS.

### Cytotoxicity Assays

Human pancreatic cancer cell lines were seeded in a 96-well plate at a density of 5x103 cells/well and allowed to adhere overnight. The culture medium was then replaced with medium containing free GEM, IgG or ICAM1 conjugated DM1, Duo, MMAE, MMAF at different drug concentrations. After cells were cultured for another 72 hours, the cytotoxicity was determined by CCK-8 assay following the vendor-provided protocol. Cells were carefully rinsed with PBS after the drug-containing medium was removed, and this was followed by adding the CCK-8 containing medium solution. The cells were then incubated with the CCK-8 medium for 4 hours. The plate was read at the absorbance wavelength of 450 nm using a microplate reader (Synergy2; BioTek, Winooski, Vt., USA). Cell viability was determined by comparing the absorbance of cells incubated with drugs to that of the control cells incubated without the presence of the drug.

### Orthotopic PDAC Mouse Models

All animal experiments were conducted following a protocol approved by the Institutional Animal Care and Use Committee (IACUC) at Boston Children's Hospital. PANC-1 cells were transfected with a plasmid expressing the luciferase and GFP genes according to the manufacturer's instructions (MGH, Boston). Successful gene transfer was confirmed 72 hours after infection, by the visualization of GFP on fluorescence microscopy. Stably transfected cells are sorted twice by flow cytometry for GFP signal using a FACSAria II flow cytometry (BD Biosciences), and maintained in DMEM-10% FBS. The orthotopic pancreatic cancer model was prepared by injecting PANC-1 cells into the pancreas of 8-week-old male athymic nude mice (Charles River; n=6 for each group) using an established surgical method. Mice were anesthetized by isofluorane (5% with O2) during the surgery. Incision was made on the left flank of the abdominal region where the pancreas is typically located, behind the middle of the spleen. The pancreas was then gently pulled out using forceps and 50 μL 1×10⁶ PANC-1 cells was carefully injected into the pancreas. After injection, the pancreas was placed back in the abdominal cavity before the abdominal muscle and the skin were closed with 4-0 Polysorb sutures and surgical staples. Treatment was started after two weeks of recovery. Animals were randomly divided into four groups (n=6): PBS control, treated with gemcitabine (GEM), treated with nontargeted IgG-DM1, and treated with ICAM1-DM1. The mice were treated through intravenous tail vein injection at a dose of 12 mg/kg mouse weight per three weeks for IgG-DM1 and ICAM1-DM1, 80 mg/kg mouse weight twice a week for GEM, while the control group received only PBS injection. In total, there were two injections with 3-week intervals for ADC treated and control groups, and 12 injections with 3- or 4-day intervals for GEM. The body weights were measured twice a week, and tumor growth was monitored using the IVIS Spectrum Imaging System (PerkinElmer) after mice received i.p. injection of D-luciferin.

### In Vivo MRI

In vivo MRI was performed on the tumor-bearing mice in two groups, which were injected intravenously with IgG-Gd and ICAM-Gd (at the dosage of 5 mg/kg mouse weight), respectively. Images were obtained at pre- and 24 hours-post injection with a 9.4 T Bruker

Horizontal Bore MRI with turbo spin echo sequence for T1- and T2-weighted MRI. The imaging parameters were as follows: repetition time (TR) of 1,523 ms, TE of 33 ms, 340 x 220 matrix, 40×28 -mm2 field of view, 180° flip angle, and 0.6-mm slice thickness for T2-weighted imaging; TR of 700 ms and TE of 22 ms for T1-weighted imaging. To quantify the signal intensity for tumor, regions of interest (ROIs) were drawn around the whole tumor at the same slice with the same imaging depth. The pixel intensity was calculated and normalized to the area of ROIs by ImageJ software.

### Histology.

The organs (liver, spleen, kidney, pancreas, heart, lung and muscle) and tumor samples were collected at the end point. Pathologies of orthotopic PANC-1 tumors treated with ICAM-DM1, IgG-DM1, GEM and PBS were investigated by H&E staining, Ki67 staining, and ICAM1 immunohistological staining. All staining was performed for the tumor slices following the standard protocol.

### Statistical Analysis

Quantitative data are presented as means±SD. Differences were compared using an unpaired t test. Statistics were performed using Microsoft Excel software. P values<0.05 were considered statistically significant.

### Examples of the Structures of the Linker and Drug in the ADCs

The linker and drug structures used in the present disclosure are provided below.

### Name: SMCC-DM1

Chemical Name: N2′-deacetyl-N2′-[3-[[1-[[4-[[-(2,5-dioxo-1-pyrrolidinyl)oxy]carbonyl]cyclohexyl]methyl]-2,5-dioxo-3-pyrrolidinyl]thio]-1-oxopropyl]-maytansine

### Chemical Structure

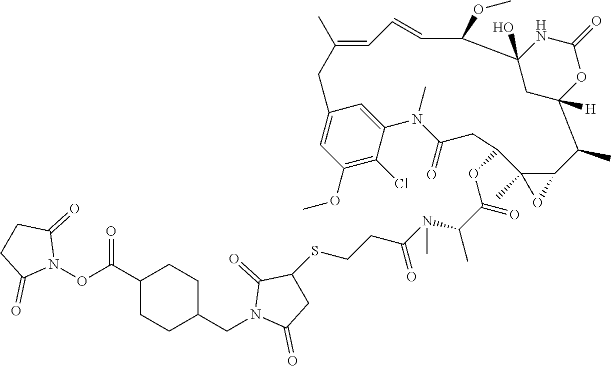

### Name: VC-MMAE (MC-VC-PAB-MMAE)

Chemical Name: 4-((S)-2-((S)-2-(6-(2,5-dioxo-2,5-dihydro-1H-pyrrol- 1-yl)hexanamido)-3-methylbutanamido)-5-ureidopentanamido)benzyl ((S)-1-(((S)-1-(((3R,4S ,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3 -methoxy-5-methyl-1-oxoheptan-4-yl)(methyl)amino)-3 -methyl-1-oxobutan-2-yl)amino)-3-methyl-1-oxobutan-2-yl)(methyl)carbamate

### Chemical Structure

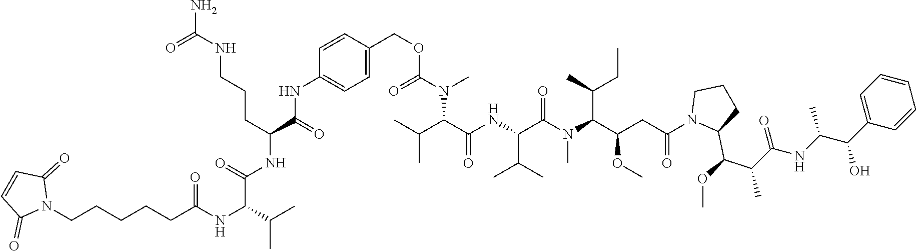 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

### Name: MC-MMAF

Chemical Name: ((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanoyl)-L-phenylalanine

### Chemical Structure

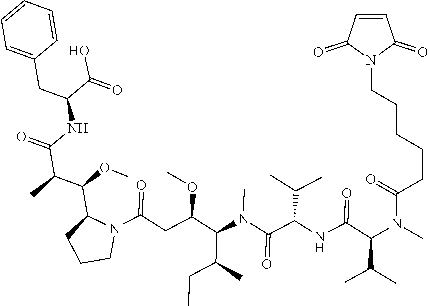 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C]

Name: Mal-PEG4-VC-PAB-DMEA-Seco-Duocarmycin Chemical Name: methyl (8S)-4-[2-[[4-[[(2S)-5-(carbamoylamino)-2-[[(2S)-2[3-[2-[2-[2-[2-[3-(2,5-dioxopyrrol-1-yl)propanoylamino]ethoxy]ethoxy]ethoxy]ethoxy]propanoylamino]-3-methylbutanoyl]amino]pentanoyl]amino]phenyl]methoxycarbonyl-methylamino]ethyl-methylcarbamoyl]oxy-8-(chloromethyl)-6-(5,6,7-trimethoxy-1H-indole-2-carbonyl)-7,8-dihydro-3H-pyrrolo[3,2-e]indole-2-carboxylate

### Chemical Structure

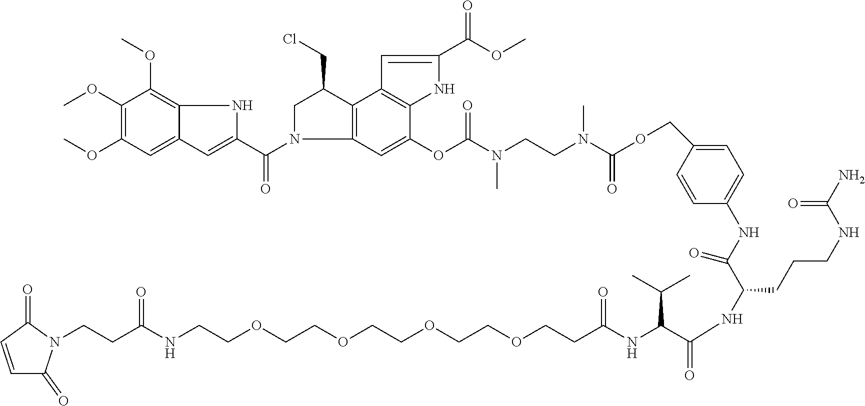

### Equivalents and Scope

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method of treating pancreatic cancer, the method comprising administering to a subject in need thereof an effective amount of an antibody drug conjugate (ADC) comprising an intercellular adhesion molecule 1 (ICAM1) antibody conjugated to a drug.

2. The method of claim 1, wherein the drug is selected from the group consisting of: N2′-Deacetyl-N2′-(3-mercapto-1-oxopropyl)mertansine (DM1), monomethyl auristatin E (MMAE), monomethyl auristatin F (MMAF), and duocarmycin.

3. The method of claim 2, wherein the drug is DM1.

4. The method of any one of claims 1-3, wherein the ICAM1 antibody and the drug is conjugated via a linker.

5. The method of claim 4, wherein the linker is a cleavable linker.

6. The method of claim 5, wherein the cleavable linker is selected from the group consisting of: N-succinimidyl 4-(2-pyridyldithio)pentanoate (SPP), N-succinimidyl 3-(2-pyridyldithio)butanoate (SPDB), Sulfo-SPDB, valine-citrulline (Val-cit), acetyl butyrate, and CL2A.

7. The method of claim 6, wherein the cleavable linker is Val-cit.

8. The method of claim 4, wherein the linker is a non-cleavable linker.

9. The method of claim 8, wherein the non-cleavable linker is a selected from the group consisting of N-succinimidyl 4-(Nmaleimidomethyl)cyclohexane-1-carboxylate (SMCC) and maleimidomethyl cyclohexane-1-carboxylate (MCC).

10. The method of claim 8, wherein the non-cleavable linker is a N-succinimidyl 4-(Nmaleimidomethyl)cyclohexane-1-carboxylate (SMCC) linker.

11. The method of any one of claims 1-10, wherein the ICAM1 antibody is selected from the group consisting of an IgG, an Ig monomer, a Fab fragment, a F(ab′)2 fragment, a Fd fragment, a scFv, a scAb, a dAb, a Fv, an affibody, a diabody, a single domain heavy chain antibody, and a single domain light chain antibody.

12. The method of any one of claims 1-11, wherein the ICAM1 antibody is Enlimomab or HCD54.

13. The method of any one of claims 1-12, wherein the ratio of the ICAM1 antibody and the drug in the ADC is 1:1 to 1:10.

14. The method of claim 13, wherein the ratio of the ICAM1 antibody and the drug in the ADC is 1:4.

15. The method of any one of claims 1-14, wherein the ADC is administered via injection.

16. The method of claim 15, wherein the injection is intravenous injection or intratumoral injection.

17. A method of treating pancreatic cancer, the method comprising administering to a subject in need thereof an effective amount of an antibody drug conjugate (ADC) comprising an intercellular adhesion molecule 1 (ICAM1) antibody conjugated to N2′-Deacetyl-N2′-(3-mercapto-1-oxopropyl)mertansine (DM1) via a N-succinimidyl 4-(Nmaleimidomethyl)cyclohexane-1-carboxylate (SMCC) linker.

18. A method of predicting the responsiveness of treatment with an ICAM1 antibody or an antibody drug conjugate (ADC) comprising an intercellular adhesion molecule 1 (ICAM1) antibody conjugated to a drug in a subject having pancreatic cancer, the method comprising:
(i) administering to the subject an effective amount of an ICAM1 antibody labeled with an imaging agent; and
(ii) visualizing the tumor via imaging;
(iii) determining the level of ICAM1 on the tumor,
wherein a higher level of ICAM1 indicates that the subject is more responsive to treatment with the ICAM1 antibody or the ADC compared to a subject having a lower level of ICAM1.

19. The method of claim 18, wherein the ICAM1 antibody in (i) is labeled with DTPA-Gd.

20. The method of claim 18 or claim 19, wherein the visualizing in (ii) is via magnetic resonance imaging (MRI).

21. The method of any one of claims 18-20, further comprising administering an effective amount of the ICAM1 antibody or the ADC to the subject predicted to be responsive to the treatment to treat the pancreatic cancer.

22. The method any one of claims 18-21, wherein the drug is selected from the group consisting of: N2′-Deacetyl-N2′-(3-mercapto-1-oxopropyl)mertansine (DM1), monomethyl auristatin E (MMAE), monomethyl auristatin F (MMAF), and duocarmycin.

23. The method of claim 22, wherein the drug is DM1.

24. The method of any one of claims 18-23, wherein the ICAM1 antibody and the drug is conjugated via a linker.

25. The method of claim 24, wherein the linker is a cleavable linker.

26. The method of claim 25, wherein the cleavable linker is selected from the group consisting of: N-succinimidyl 4-(2-pyridyldithio)pentanoate (SPP), N-succinimidyl 3-(2-pyridyldithio)butanoate (SPDB), Sulfo-SPDB, valine-citrulline (Val-cit), acetyl butyrate, and CL2A.

27. The method of claim 24, wherein the cleavable linker is Val-cit.

28. The method of claim 24, wherein the linker is a non-cleavable linker.

29. The method of claim 28, wherein the non-cleavable linker is a selected from the group consisting of N-succinimidyl 4-(Nmaleimidomethyl)cyclohexane-1-carboxylate (SMCC) and maleimidomethyl cyclohexane-1-carboxylate (MCC).

30. The method of claim 29, wherein the non-cleavable linker is a N-succinimidyl 4-(Nmaleimidomethyl)cyclohexane-1-carboxylate (SMCC) linker.

31. The method of any one of claims 18-30, wherein the ICAM1 antibody is selected from the group consisting of an IgG, an Ig monomer, a Fab fragment, a F(ab′)2 fragment, a Fd fragment, a scFv, a scAb, a dAb, a Fv, an affibody, a diabody, a single domain heavy chain antibody, and a single domain light chain antibody.

32. The method of any one of claims 18-30, wherein the ICAM1 antibody is Enlimomab or HCD54.

33. The method of any one of claims 18-32, wherein the ratio of the ICAM1 antibody and the drug in the ADC is 1:1 to 1:10.

34. The method of claim 33, wherein the ratio of the ICAM1 antibody and the drug in the ADC is 1:4.
